(19) United States
(12) Patent Application Publication
(10) Pub. No.: US 2025/0064894 A1
(43) Pub. Date: Feb. 27, 2025

(54) METHODS AND COMPOSITIONS FOR ENHANCEMENT OF TUMOR IMMUNOGENICITY AND STIMULATING ANTI-TUMOR IMMUNE RESPONSES IN AN ANIMAL

(71) Applicants: WASHINGTON UNIVERSITY, St. Louis, MO (US); BOLDER BIOTECHNOLOGY, INC., Boulder, CO (US)

(72) Inventors: Matthew J. CHRISTOPHER, St. Louis, MO (US); Michael P. RETTIG, St. Louis, MO (US); Allegra PETTI, St. Louis, MO (US); John F. DIPERSIO, St. Louis, MO (US); Timothy J. LEY, St. Louis, MO (US); George N. COX, Louisville, CO (US)

(21) Appl. No.: 18/726,712

(22) PCT Filed: Jan. 6, 2023
(86) PCT No.: PCT/US23/60259

§ 371 Date: Jul. 3, 2024

Related U.S. Application Data
(60) Provisional application No. 63/297,157, filed on Jan. 6, 2022.

(51) Int. Cl. A61K 38/21; A61K 39/395; A61P 35/00

(52) U.S. Cl. CPC A61K 38/217; A61K 39/39558; A61P 35/00

(57) ABSTRACT

Provided herein are methods and compositions for enhancement of tumor immunogenicity and for stimulating anti-tumor immune responses in an animal.

### CROSS-REFERENCE TO RELATED APPLICATIONS

This application claims the benefit of priority under 35 U.S.C. § 119 (e) to U.S. Provisional Patent Application No. 63/297,157 filed Jan. 6, 2022. The entire disclosure of which is incorporated herein by reference.

### GOVERNMENT RIGHTS

This invention was made with government support under grant 1R43 AI157684-01A1 awarded by the National Institutes of Health and the National Institute of Allergy and Infectious Disease and project number 5P01CA101937 award by the National Institutes of Health and the National Cancer Institute. The government has certain rights in the invention.

### REFERENCE TO A SEQUENCE LISTING

This application contains a Sequence Listing submitted electronically as an XML file. The text file, named “4152-23-PCT.xml”, has a size in bytes of 4000 B, and was recorded on Jan. 4, 2023. The information contained in the XML file is incorporated herein by reference in its entirety pursuant to 37 CFR § 1.52 (e) (5).

### BACKGROUND OF THE INVENTION

Cancer is the second leading cause of death in the U.S., second only to heart disease. Approximately 600,000 people die from cancer each year in the U.S., with the most common cancer types being lung, colon, pancreas, breast and prostate. Tumors have evolved several mechanisms to escape immune killing such as decreased major histocompatibility complex class I (MHC-I) and major histocompatibility complex class II (MHC-II) expression, which reduces tumor antigen presentation and recognition and killing by host immune cells. Up to half of acute myelogenous leukemias (AML) that relapse following hematopoietic stem cell transplant (HCT) express much lower levels of MHC-II proteins and other proteins involved in antigen processing and presentation compared to the same tumors analyzed prior to relapse (Christopher et al., 2018, N Engl J Med. 379 (24): 2330-2341; Toffalori et al. 2019, Nature Medicine 25, 603-611). Many triple negative breast cancer cells (TNBCs) exhibit low MHC-II expression, which correlates with poor survival prognosis (Forero et al., 2016, Cancer Immunol Res 4:390-399). Higher TNBC MHC-II expression correlates with good survival prognosis. Thus, there is a great need for methods and drugs capable of reversing the phenotypic changes (low MHC-II expression) in relapsed AML cells that allow these cancer cells to escape killing by host immune cells. Similarly, there is a great need for methods and drugs capable of increasing MHC-II expression in TNBCs, and other cancer types to increase their immunogenicity and killing by host immune cells. Low MHC-II expression by some relapsed AML cells can be reversed by treating the relapsed AML cells in vitro with interferon gamma (IFNg) (Christopher et al., 2018, N Engl J Med. 379 (24): 2330-2341). This finding indicates that decreased expression (down regulation) of MHC-II proteins in relapsed AML cells results from reversible epigenetic changes rather than permanent genetic mutations. Some low MHC II-expressing TNBC cell lines can be induced to express MHC-II proteins and other proteins involved in antigen processing and presentation by treating the TNBC cells in vitro with IFNg (Forero et al., 2016, Cancer Immunol Res 4:390-399), indicating that low MHC-II expression is a reversible phenotype.

Another mechanism commonly used by tumors to escape immune killing is expression of cell surface proteins such as PD-L1 (programmed death ligand 1; CD274), which inhibit host immune cell responses and create an immunosuppressive tumor microenvironment. Blocking PD-L1 interaction with its cognate receptor PD-1 (programmed cell death protein 1; CD279) on immune cells using anti-PD-L1 or anti-PD-1 monoclonal antibodies (mAbs) overcomes the immunosuppressive effects of PD-L1 and allows host immune cells to become activated to kill tumor cells. Anti-PD-L1/anti-PD-1 mAb therapies have revolutionized cancer treatment paradigms and greatly improved cancer survival rates. However, these mAbs are effective for only about 20% of tumors. Thus, there is a great need for new drugs and methods to treat the remaining 80% of tumors that are non-responsive to anti-PD-L1/anti-PD-1 therapies.

### SUMMARY

One embodiment is a method and/or use for inhibiting growth of a tumor in an animal having a tumor comprising administering to the animal one or more effective doses of an interferon gamma (IFNg) analog, wherein the IFNg analog increases major histocompatibility complex (MHC-II) protein expression, tumor cell major histocompatibility complex (MHC-I) protein expression and/or tumor cell programmed death ligand 1 (PD-L1) protein expression. Optionally, the animal may be administered one or more effective doses of an anti-PD-L1 antibody or an anti-programmed cell death protein 1 (PD-1) antibody, a checkpoint inhibitor antibody, or a T cell agonist drug.

In one aspect, the animal is further administered one or more effective doses of an anti-PD-L1 antibody or an anti-PD-1 antibody, wherein the IFNg analog induces expression of PD-L1 in the tumor cell, and wherein tumor growth inhibition with the combination of the IFNg analog and the anti-PD-L1 antibody or the anti-PD-1 antibody is greater than the tumor growth inhibition obtained by treatment with the IFNg analog alone or the anti-PD-L1 antibody alone or the anti-PD-1 antibody alone.

Another embodiment disclosed herein is a method and/or use for stimulating an anti-tumor immune response in an animal having a tumor comprising administering to the animal one or more effective doses of an IFNg analog. In one aspect, the animal is further administered one or more effective doses of an anti-PD-L1 antibody or an anti-PD-1 antibody, wherein the IFNg analog induces expression of PD-L1 in tumor cells of the animal, and wherein the anti-tumor immune response obtained with the combination of the IFNg analog and the anti-PD-L-1 antibody or anti-PD-1 antibody is greater than the anti-tumor immune response obtained by treatment with the IFNg analog alone or the anti-PD-L1 antibody alone or the anti-PD-1 antibody alone. In one aspect, the IFNg analog increases expression of PD-L1, MHC Class II proteins, and/or MHC Class I proteins in tumor cells of the animal.

Another aspect of the invention is a method and/or use for increasing the number of natural killer (NK) cells in an animal, comprising administering to the animal one or more effective doses of an interferon gamma analog.

Another embodiment disclosed herein is a method and/or use for stimulating a graft versus leukemia anti-tumor immune response in an animal comprising administering to an animal with a leukemia an IFNg analog that stimulates increased expression of MHC-II proteins or MHC-I proteins in the leukemia cells of the animal.

Another embodiment is a method and/or use of increasing the number of natural killer (NK) cells in an animal, comprising administering to the animal one or more effective doses of an IFNg analog.

In one aspect of any of the methods and/or uses disclosed herein, the animal may be administered one or more effective doses of an anti-PD-L1 antibody, an anti-PD-1 antibody, a checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-cytotoxic T-lymphocyte Antigen 4 (anti-CTLA4) antibody, an anti-lymphocyte-activator gene 3 (anti-LAG-3) antibody, an anti-T-cell immunoglobulin mucin-3 antibody (anti-TIM 3 antibody), an anti-tissue inhibitor of matrix metalloproteinase antibody (anti-TIMP antibody), and an anti-T-cell immunoreceptor with Ig and ITIM domains antibody (anti-TIGIT antibody). Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist (also known as tumor necrosis factor ligand superfamily member 9), and a CD40 agonist. Preferably the T cell agonist protein is an antibody.

The methods and/or uses described herein are generally applicable to all types of tumors and cancer types, including but not limited to leukemias, including acute myelogenous leukemias (AML, also referred to as acute myeloid leukemia), lymphomas, myelomas, glioblastomas, melanomas, lung cancers, kidney cancers, liver cancers, pancreatic cancers, breast cancers, triple negative breast cancers, colon cancers, bladder cancers, fibrosaracoma cancers, gastric cancers, esophageal cancers, prostate cancers, and bone cancers. In one aspect, the leukemia is acute myelogenous leukemias (AML). The methods and/or uses may be particularly useful for treating tumor cells and cancer cells that express low or no levels of MHC-I, MHC-II, or PD-L1 proteins. One preferred tumor cell type is AML which is resistant to anti-PD-1 antibody therapy or anti-PD-L1 antibody therapy. One aspect, the tumor is a T cell resistant tumor. One aspect, the tumor is a relapsed tumor. One preferred tumor cell type is an AML tumor that has relapsed, including relapsed after HCT. More preferably, the tumor type is an AML tumor that has relapsed after HCT and expresses low or no levels of one or more of the following proteins: MHC-I proteins, MHC-II proteins, or PD-L1 proteins. Most preferably, the relapsed AML tumor cells express lower levels of one or more proteins selected from the group consisting of MHC-I proteins, MHC-II proteins, or PD-L1 proteins compared to the patient's AML cells prior to relapse. Another preferred tumor type is a breast cancer tumor, more preferably a triple negative breast cancer tumor, and most preferably a triple negative breast cancer tumor that expresses low or no levels of one or more of the proteins selected from the group consisting of MHC-I proteins, MHC-II proteins, or PD-L1 proteins.

The methods and/or uses disclosed herein will also be useful for treating tumors and cancers in an animal in combination with other chemotherapeutic agents, anti-cancer drugs, checkpoint inhibitor antibodies, checkpoint inhibitor monoclonal antibodies, cell therapies, donor immune cells, donor lymphocytes, donor T lymphocytes, donor white blood cells, donor macrophages, donor NK cells, donor B cell lymphocytes, donor dendritic cells, engineered anti-tumor T cell therapies, engineered anti-tumor macrophage cell therapies, anti-tumor NK cell therapies, anti-tumor B cell therapies, anti-tumor dendritic cell therapies and combinations thereof.

In one aspect of any of the methods and/or uses disclosed herein, the IFNg analog is a long-acting IFNg analog, wherein the long acting INFg analog is modified with a polyethylene glycol (PEG).

In one aspect the INFg analog is modified with a PEG at a lysine residue of the protein.

In one aspect, the INFg analog is modified with a PEG at the amino-terminus of the protein.

In one aspect, the INFg analog is modified with a PEG at a sugar group in the protein.

In one aspect, the IFNg analog is an isolated cysteine variant of human IFNg (SEQ ID NO: 1), wherein a cysteine residue is substituted for at least one amino acid selected from the group consisting of: D63, Q64, S65, I66, Q67, V99, V100, T101, D102 and L103.

In one aspect, the isolated cysteine variant further comprises deletion or substitution of Q1 of SEQ ID NO:1 by a non-glutamine amino acid, and/or deletion or substitution of D2 of SEQ ID NO:1 by a non-aspartic amino acid. In one aspect, the variant is modified with a cysteine reactive moiety, wherein said cysteine reactive moiety is a PEG.

In one aspect, the INFg analog is a cysteine variant of human IFNg (SEQ ID NO: 1) comprising at least one cysteine residue inserted in at least one region of IFNg selected from the group consisting of: the region preceding the first amino acid of SEQ ID NO: 1, and the region following the last amino acid of SEQ ID NO:1.

In one aspect, the IFNg analog is a cysteine variant of human IFN-g (SEQ ID NO: 1) comprising at least one cysteine residue substituted for at least one amino acid located in the C-D loop of IFN-g. In one aspect, the IFNg analog further comprises at least one cysteine residue substituted for at least one amino acid located in the region preceding helix A of IFNg. In still another aspect, the IFNg analog further comprises at least one cysteine residue substituted for at least one amino acid located in the B-C loop of IFNg. In yet another aspect, the IFNg analog further comprises at least one cysteine residue substituted for at least one amino acid located in the D-E loop of IFNg. In still yet another aspect, the variant is modified with a cysteine reactive moiety, wherein said cysteine reactive moiety is a PEG.

In one aspect of any of the methods and/or uses disclosed herein, the IFNg analog is human IFNg (SEQ ID NO:1) or an analog of human IFNg (SEQ ID NO:1).

### BRIEF DESCRIPTION OF THE DRAWINGS

FIG. 1 shows inhibition of murine B16BL6 tumor cell growth in vitro by wild-type (WT) mIFNg and PEG mIFNg (S66C). IFNg (S66C) and PEG mIFNg (S66C) gave similar inhibition curves. B16BL6 tumor cells (10,000 per well) were incubated with the indicated amounts of WT IFNg or PEG-IFNg (S66C) for 4 days at 37° C. An MTS dye solution was then added to the plates and absorbance, which is proportional to cell number, was read 1 h later at 490 nm using a plate reader. Data are means±SD of triplicate wells for each protein dilution.

FIG. 2 shows the results of xenograft mice engrafted with a human patient's AML tumor sample expressing low MHC-II that were treated with IFNg, PEG IFNg, or vehicle for two weeks, dosing 3 times per week on Mondays, Wednesdays, and Fridays. Mice were sacrificed and MHC-II expression on human AML cells was measured by flow cytometry. Shown is mean fluorescence intensity of each group (N=3 for each group).

FIG. 3 shows that lethally irradiated mice treated with PEG mIFNg four days after receiving allogeneic T cell depleted bone marrow transplantation and allogeneic T cell transplantation on the same day die by day 10. Lethally irradiated B6 recipient mice received 4×1⁰⁶ allogeneic T cell depleted BALB/c bone marrow (BM) with or without 2.5×10⁶ allogeneic BALB/c T cells on the day of transplant. Recipient mice were then treated with phosphate buffered saline (PBS) or 15 μg per mouse of PEG-mIFNg by subcutaneous injection on days 4, 6, and 8 after transplantation. Mice receiving allogeneic T cells and PEG-mIFNg died on day 10 after transplant while the BM only cohort treated with PEG-mIFNg all survived.

FIG. 4 shows that delaying allogeneic T cell transplantation and PEG mIFNg treatment until ten days after allogeneic T cell depleted bone marrow transplantation in lethally irradiated mice leads to long-term survival. Lethally irradiated B6 recipient mice received 4×10⁶ allogeneic T cell depleted BALB/c bone marrow (BM) on the day of transplant. Ten days after transplantation, recipient mice were either left untreated or injected with 2.5×10⁶ allogeneic BALB/c T cells. Mice were then treated with phosphate buffered saline (PBS) or 15, 3.8, or 1.8 μg per mouse of PEG-mIFNg by subcutaneous injection on days 10, 12, 14, 16, 18, and 20 after transplant. All mice receiving allogeneic T cells and PEG-mIFNg survived 96 days after transplant.

FIG. 5 shows selection of allo-T resistant AML1 cells by serial transplantation following relapse in allo-HSCT mice. Lethally irradiated B6 recipient mice were transplanted with syngeneic B6 AML1 tumors and allogeneic T cell depleted bone marrow (BM) from 129 SVJ mice in the presence or absence (BM only) of allogeneic 129 SVJ wild type T cells. AML1 cells were harvested from relapsing mice and re-transplanted into new B6 recipient mice in serial transplantation. The graph shows percent survival over time. In vivo allogeneic T cell resistance is demonstrated by the AML1 cells treated with allo-T cells in the tertiary HSCT transplant dying at about the same time as the no allo-T cell treated control mice. The data in the graph represents results from two independent experiments.

FIG. 6 shows that IFNg re-sensitizes allo-T cell resistant tumors to killing by day 10 donor lymphocyte infusion (allo-T cells) and extends survival. Lethally irradiated B6 recipient mice were transplanted with T cell depleted BM from 129 SVJ mice in the presence or absence of allogeneic wild type 129 SVJ T cells with and without IFNg treatment. The IFNg groups received six doses of 15 or 3.8 μg PEG mIFNg (3 times per week (on Monday, Wednesday and Friday of each week) for 2 weeks) via the sc route. The graph shows probability of survival over time. IFNg alone extended survival modestly compared to no IFNg controls. Mice treated with the combination of IFNg and allo-T cells had significantly extended survival compared to mice treated with IFNg only or allo-T cells only. The data in the graph represents results from two independent experiments. TCD BM, T cell depleted bone marrow.

FIG. 7 shows that IFNg treatment clears the green fluorescent protein (GFP) positive AML tumor blast cells from mice. Lethally irradiated B6 recipient mice were transplanted with 129 SVJ allogeneic BM (T cell depleted) in the presence or absence of allogeneic wild-type T cells from 129 SVJ mice with and without IFNg treatment. The IFNg groups received 6 doses of 15 or 3.8 μg PEG IFNg (3 times/week×2 weeks) via the sc route. On day 30 post allo-HSCT flow cytometry was done on peripheral blood CD45 positive cells of the different treatment groups, measuring % GFP positive cells as an indicator of AML1 tumor burden. The results showed that GFP positive AML blasts were significantly reduced or undetectable in peripheral blood of mice treated with PEG mIFNg and allo-T cells compared to mice treated with allo-T cells only, PEG IFNg only, or vehicle only. Each dot represents results from a single mouse. The bar height represents the group mean.

### DETAILED DESCRIPTION OF THE INVENTION

Described herein are methods and/or uses for enhancing anti-tumor immunity and stimulating an anti-tumor immune response in an animal. Non-responsiveness to anti-PD-L1 mAbs is due in many cases to low tumor cell PD-L1 expression. A tumor in an animal is considered non-responsive to anti-PD-1 or anti-PD-L1 therapies if the tumor is not killed following treatment of the animal with anti-PD-1 or anti-PD-L1 therapies, if growth of the tumor is not inhibited following treatment of the animal with anti-PD-1 or anti-PD-L1 therapies, or if treatment of the animal with a tumor with anti-PD-1 or anti-PD-L1 therapies does not improve survival of the animal with a tumor.

As disclosed herein, the inventors discovered that IFNg is able up-regulate PD-L1 expression (as well as MHC-I expression and MHC-II expression) on some tumor cells that normally display low PD-L1 (and/or low MHC-I expression and/or low MHC-II expression) levels in vitro, and thus has the potential to increase tumor antigen presentation and immune killing in vivo. However, since PD-L1 is immunosuppressive there is a concern that use of IFNg to treat an animal with a tumor might inhibit the animal's anti-tumor immune response. One solution to this problem is to treat the animal with a tumor with both IFNg and an anti-PD-L1 therapy or an anti-PD-1 therapy.

The inventors believe that using IFNg and other drugs capable of increasing tumor cell PD-L1 expression will sensitize the tumor to anti-PD-L1 or anti-PD-1 therapies, resulting in superior tumor growth inhibition with the combination therapy. Superior tumor growth inhibition in an animal can be measured in multiple ways, including, but not limited to, demonstrating decreased tumor volumes or tumor weights in an animal with a tumor, or demonstrating increased survival, extended survival, or prolonged survival in an animal with a tumor.

In addition to stimulating tumor cell MHC-I, MHC-II, and PD-L1, IFNg also directly inhibits growth of many tumor cell types in vitro (Fam et al. 2014, Journal of Interferon and Cytokine Research 34:759-768; Wall et al., 2003, Gynecol Oncol 88:149-151). However, tumor cell growth inhibition generally requires continuous exposure of the tumor cells to IFNg for several days, which is easy to maintain in vitro but difficult in vivo due to IFNg's short half-life and poor bioavailability. The inventors hypothesized that IFNg's short half-life and poor bioavailability would also limit the protein's ability to up-regulate tumor cell MHC-I, MHC-II, and PD-L1 expression in vivo and limit the protein's utility as an anti-cancer therapeutic. As disclosed herein the inventors confirmed this hypothesis by engineering a long-acting IFNg analog with improved pharmacokinetic properties and demonstrating that the protein is significantly more effective than IFNg at inhibiting tumor growth in an animal and also more effective than IFNg at up-regulating tumor cell MHC-II expression in an animal. The inventors also discovered that the long-acting IFNg protein is effective at increasing tumor cell PD-L1, MHC-I, and MHC-II expression when administered to an animal with a tumor expressing low levels of these proteins. The inventors further discovered that the long-acting IFNgs are effective at increasing MHC-I, MHC-II and PD-L1 expression on host cells in an animal.

Still further, the inventors also discovered that IFNg analogs, particularly a long-acting IFNg analog, is able to re-sensitize T cell resistant AML tumor cells to allogeneic T cell killing in mice, as evidenced by extended survival (as measured by increased median survival time) of mice treated with the combination of the longer-acting IFNg plus donor allogeneic T cells compared to mice treated with the longer-acting IFNg protein alone or the donor allogeneic T cells alone. A T cell resistant tumor is a tumor in an animal that is not killed by T cells or a tumor whose growth in an animal is not inhibited by T cells.

The methods disclosed herein entail using an interferon gamma analog to stimulate increased tumor cell expression of MHC-I, MHC-II or PD-L1 proteins. The methods also entail using an interferon gamma analog to stimulate increased expression of MHC-I, MHC-II or PD-L1 proteins in cells of an animal. The methods also entail using an interferon gamma analog in combination with donor lymphocytes, preferably allogeneic donor lymphocytes, to enhance anti-tumor immunity and to stimulate an anti-tumor immune response in an animal. A preferred agent for stimulating MHC-I protein expression or MHC-II protein expression or PD-L1 protein expression in a tumor cell or in an animal's cells is an interferon gamma analog. A preferred interferon analog is a human interferon gamma analog (SEQ ID NO:1), such as Actimmune (Horizon Pharma). A more preferred interferon gamma analog is a long-acting interferon gamma analog, which is an interferon gamma analog that has a longer circulating half-life (terminal half-life) in an animal compared to interferon gamma following administration to the animal. A preferred long-acting interferon gamma analog is an interferon analog modified with polyethylene glycol (PEG). Different PEGs can be used to modify interferon gamma preferentially at its amino-terminus, or at lysine residues, at one or more non-native or non-natural amino acids incorporated into the interferon gamma analog by addition at the amino-terminus or carboxy terminus of the protein or by substitution for an one or more amino acids in the interferon gamma protein, or at sugar groups in the protein. Another preferred method to prepare a PEGylated interferon gamma analog is to modify a cysteine residue in the interferon gamma analog. Most animal interferon gamma proteins, including human interferon gamma, do not contain any native cysteine residues. Methods for creating biologically active cysteine variant interferon gamma proteins are described in U.S. Pat. Nos. 9,296,804, 8,618,256, 8,617,531, 7,964,184 and 7,959,909 (each are herein incorporated by reference). A cysteine residue can be added to human interferon gamma by insertion preceding the first amino acid of the mature protein or by insertion following the last amino acid of the mature protein. A cysteine residue can be added to human interferon gamma by insertion between two adjacent amino acids within the primary amino acid sequence of interferon gamma as long as the biological activity or protein's conformation is not significantly affected by the cysteine insertions. A cysteine residue can be substituted for at least one amino acid in human interferon gamma having SEQ ID NO:1 selected from the group consisting of: Q1, D2, P3, N16, A17, G18, H19, S20, D21, V22, A23, D24, N25, G26, K37, E38, E39, S40, D63, Q64, S65, I66, Q67, N83, S84, N85, K86, N97, Y98, S99, V100, T101, D102, L103, P122, A123, A124, K125, T126, G127, K128, R129, K130, R131, S132, Q133, M134, L135, F136, R137, G138, R139, R140, A141, S142, and Q143. Preferred human interferon gamma cysteine variant proteins contain a cysteine substitution for the L103 amino acid (referred to as L103C) or a cysteine substitution for the Q67 amino acid (referred to as Q67C). These interferon gamma cysteine variants can be incorporated into the native interferon gamma sequence or in variants in which Q1 is deleted, or D2 is deleted, or Q1 and D2 are deleted or changed to non-glutamine or non-aspartic acids, respectively. Possible substitutions include amino acids such as alanine, arginine, aspartic acid (for Q1 only), asparagine, glutamic acid (for D2 only), glycine, histidine, isoleucine, leucine, lysine, methionine, phenylalanine, proline, serine, threonine, tryptophan, tyrosine, or valine). These interferon gamma cysteine variants also can be incorporated into the native interferon gamma sequence or in variants comprising a methionine inserted preceding the Q1 amino acid or a methionine inserted preceding the D2 amino acid if the Q1 amino acid is deleted.

Preferably, the long-acting interferon gamma analog is able to maintain a plasma level equal to or greater than 0.01 ng/ml for at least 10 h, and more preferably 24 h following administration to the animal with a tumor. More preferably, the long-acting interferon gamma analog is able to maintain a plasma level equal to or greater than 0.05 ng/ml for at least 10 h, and more preferably 24 h following administration to the animal with a tumor. Even more preferably, the long-acting interferon gamma analog is able to maintain a plasma level equal to or greater than 0.1 ng/ml for at least 10 h, and more preferably 24 h following administration to the animal with a tumor. Even more preferably, the long-acting interferon gamma analog is able to maintain a plasma level equal to or greater than 0.5 ng/ml for at least 10 h, and more preferably 24 h following administration to the animal with a tumor. Even more preferably, the long-acting interferon gamma analog is able to maintain a plasma level equal to or greater than 1 ng/ml for at least 10 h, and more preferably 24 h following administration to the animal with a tumor. Even more preferably, the long-acting interferon gamma analog is able to maintain a plasma level equal to or greater than 5 ng/mL for at least 10 h, and more preferably 24 h following administration to the animal with a tumor. Even more preferably, the long-acting interferon gamma analog is able to maintain a plasma level equal to or greater than 10 ng/mL for at least 10 h, and more preferably 24 h following administration to the animal with a tumor.

The methods also encompass use of viruses engineered to express interferon gamma following administration to an animal with a cancer disease. Expression of interferon gamma by the virus will result in higher and longer-lasting plasma levels of interferon gamma compared to administration of interferon gamma itself.

A further embodiment of the invention is a method for using the interferon gamma analog to treat a cancer disease, which herein is used interchangeably with the term tumor disease, in an animal, the method comprising administering the interferon analog to an animal with a cancer disease treatable with the interferon analog. Cancer diseases that may be especially amenable to treatment with the interferon gamma analog include cancers that express low levels of MHC-I, MHC-II, or PD-L1 proteins. The interferon gamma analog may be administered to the animal alone or in combination with other anti-cancer drugs, chemotherapeutic agents, cell therapies including but not limited to donor lymphocytes, donor T lymphocytes, donor white blood cells, donor macrophages, donor NK cells, donor B cell lymphocytes, donor dendritic cells, and engineered anti-tumor T cell, B cell, macrophage, dendritic cell, and NK cell therapies. Preferred anti-cancer drugs include checkpoint inhibitor drugs, including checkpoint inhibitor antibodies such as anti-PD-L1 and anti-PD-1 monoclonal antibodies.

A further embodiment of the invention is a method for treating a T cell mediated disease in an animal, the method comprising administering the interferon gamma analog to an animal with a T cell mediated disease. The T cell mediated disease can be an autoimmune disease, an inflammatory disease, or a cancer disease. The interferon gamma analog can inhibit or stimulate T cell proliferation, or stimulate or inhibit T cell effector functions. Examples of T cell effector functions that can be stimulated or inhibited by the composition include but are not limited to macrophage activation, B cell activation, B cell differentiation, stimulation of B cell antibody secretion, target cell killing, secretion of cytotoxic effector molecules such as perforins, granzymes, and fas ligand, and secretion of cytokines such as interferon gamma, tumor necrosis factor alpha, tumor necrosis factor beta, GM-CSF, CD40 ligand, fas ligand, IL-2, IL-3, IL-4, IL-5, IL-10, eotaxin, and transforming growth factor beta. Target cells killed by T cells or macrophages activated by T cells include but are not limited to cancer cells, virus infected cells, bacteria infected cells, fungi infected cells and other microbe infected cells. Inappropriate activation of T cells may result in killing or damage to healthy cells, resulting in an autoimmune disease.

A further embodiment of the invention is a method for treating a white blood cell related disease in an animal, the method comprising administering the interferon gamma analog to an animal with a white blood cell related disease. White blood cells include but are not limited to monocytes, macrophages, dendritic cells, microglia, T cells, B cells, and natural killer (NK) cells. The white blood cell related disease can be an autoimmune disease, an inflammatory disease, a cancer disease, or an immune suppressive disease. The interferon gamma analog can inhibit or stimulate white blood cell proliferation, increase or decrease white blood cell numbers, or inhibit or stimulate white blood cell effector functions. Examples of white blood cell effector functions that can be inhibited or stimulated by the interferon gamma analog include but are not limited to the T cell effector functions described above, as well as macrophage differentiation, macrophage activation, dendritic cell differentiation, dendritic cell activation, NK cell differentiation, NK cell activation, T cell differentiation, T cell activation, B cell differentiation, B cell activation, killing of cancer cells, killing of virus infected cells, killing of bacteria infected cells, killing of fungi infected cells, killing of other microbe infected cells, secretion of cytokines or other bioactive molecules, antigen processing, antigen presentation, activation of T cells, activation of B cells, activation of NK cells, stimulation of T cell immune responses, stimulation of B cell immune responses, activation of NK cells, stimulation of NK cell immune responses, stimulation of macrophage immune responses, and stimulation of tissue repair.

In one aspect, the stimulating agent is an interferon gamma analog. In another aspect, the interferon analog comprises a free cysteine residue. In still another aspect, the protein is a human interferon gamma cysteine variant. In one aspect, the human interferon gamma cysteine variant is selected from the group consisting of: (a) a human interferon gamma cysteine variant wherein a cysteine residue is inserted preceding the first amino acid of the mature protein; (b) a human interferon cysteine variant wherein a cysteine residue is inserted following the last amino acid of the mature protein; and (c) a human interferon gamma cysteine variant wherein a cysteine residue is substituted for at least one amino acid in human interferon gamma (SEQ ID NO:1) selected from the group consisting of: Q1, D2, P3, N16, A17, G18, H19, S20, D21, V22, A23, D24, N25, G26, K37, E38, E39, S40, D63, Q64, S65, I66, Q67, N83, S84, N85, K86, N97, Y98, S99, V100, T101, D102, L103, P122, A123, A124, K125, T126, G127, K128, R129, K130, R131, S132, Q133, M134, L135, F136, R137, G138, R139, R140, A141, S142, and Q143. In yet another aspect, the interferon gamma cysteine variant comprises a cysteine residue substituted for leucine at position 103 of SEQ ID NO:1. In yet another aspect, the interferon gamma cysteine variant comprises a cysteine residue substituted for glutamine at position 67 of SEQ ID NO:1. In still another aspect, the interferon gamma cysteine variant further comprising a deletion of the glutamine-1 amino acid of SEQ ID NO:1.

The PEG may be any type of PEG known in the art, including but not limited to linear, branched, forked, and multi-armed PEGs. The PEG may vary in size from 2 kDa to greater than 40 kDa. The PEG may have one or more reactive groups to facilitate attachment of the PEG to one or more amine groups in a protein, to one or more cysteine residues in a protein, to one or more sugar groups in a protein, or to one or more non-natural amino acids incorporated into a protein.

Other compounds that may be used to attach to the interferon gamma analog to increase the protein's half-life include, but are not limited to peptides, proteins, sugars, lipids, nucleic acids, and albumin binding moieties. The half-life of interferon gamma may also be increased by fusion of the interferon gamma analog to another protein domain to create a fusion protein.

The IFNg analogs of the present invention are preferably administered in a composition. Compositions can include an IFNg analog of the invention and any other suitable pharmaceutically acceptable carrier, as well as, in some aspects, additional components that may be useful in the treatment of a give disease or condition. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably any site where the IFNg analog will provide a detectable effect as compared to in the absence of the IFNg analog, and includes a disease site or a site of cell types to be contacted with the IFNg analog. Preferred pharmaceutically acceptable carriers are capable of maintaining the IFNg analog of the present invention in a form that, upon arrival of the IFNg analog at the cell target in a culture or in patient, the IFNg analog is capable of interacting with its target (e.g., tumor cell).

Suitable excipients of the present invention include excipients or formularies that transport or help transport, but do not specifically target a composition to a cell or area (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises an IFNg analog of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible). In the event that an IFNg analog of the invention is administered as a recombinant nucleic acid molecule encoding the cysteine mutein (e.g., gene therapy or genetic immunization), suitable carriers include, but are not limited to liposomes, viral vectors or other carriers, including ribozymes, gold particles, poly-L-lysine/DNA-molecular conjugates, and artificial chromosomes. Natural lipid-containing carriers include cells and cellular membranes. Artificial lipid-containing carriers include liposomes and micelles.

An effective dose of IFNg, for example, is an amount that would cause the desired therapeutic effect in an animal while minimizing undesired side effects. In various embodiments, an effective dose and/or effective dose amount of IFNg described herein can substantially inhibit growth of a hematological cancer or a solid tumor, slow the progress of a hematological cancer or a solid tumor, or limit the development of a hematological cancer or a solid tumor. An effective dose and/or effective dose amount of IFNg described herein can increase expression of MHC I proteins, MHC II proteins, or PD-L1 proteins in tumor cells in an animal or in the animal's cells. In one aspect of the invention an effective dose of an IFNg analog of the present invention is at least about 0.01 micrograms per kg of the animal to which the IFNg analog is administered, and in other aspects, at least about 0.1 micrograms/kg, at least about 0.2 micrograms/kg, at least about 0.5 micrograms/kg, at least about 1 micrograms/kg, at least about 5 micrograms/kg, at least about 10 micrograms/kg, at least about 25 micrograms/kg, at least about 50 micrograms/kg, at least about 75 micrograms/kg, at least about 100 micrograms/kg, at least about 200 micrograms/kg, at least about 300 micrograms/kg, at least about 400 micrograms/kg, at least about 500 micrograms/kg, at least about 750 micrograms/kg, at least about 1 mg/kg, or at least about 5 mg/kg. The effective dose of the interferon gamma analogs of the present invention may be administered to the animal daily, multiple times per day, every other day, 3 times per week, once weekly, once every 2 weeks, once every 3 weeks, once every 4 weeks, or by using combinations of the above dosing schedules. In one aspect, the effective dose is a single dose.

As discussed above, a therapeutic composition of the present invention is administered to a patient in a manner effective to deliver the composition to a cell, a tissue, and/or systemically to the patient, whereby the desired result is achieved as a result of the administration of the composition. Suitable administration protocols include any in vivo or ex vivo administration protocol. The preferred routes of administration will be apparent to those of skill in the art, depending on the type of condition to be prevented or treated; whether the composition is nucleic acid based or protein based; and/or the target cell/tissue. For proteins or nucleic acid molecules, preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. Routes useful for deliver to mucosal tissues include, bronchial, intradermal, intramuscular, intranasal, other inhalatory, rectal, subcutaneous, topical, transdermal, vaginal and urethral routes. Combinations of routes of delivery can be used and in some instances, may enhance the therapeutic effects of the composition. Particularly preferred routes of delivery include subcutaneous and intravenous delivery. These routes of administration can be used to administer the interferon gamma analog. These routes of administration can be used to administer any anti-cancer drugs or chemotherapeutic agents used in combination with the interferon gamma analog.

Any anti-cancer drugs used in combination with the interferon analog can be administered to the animal using the drug's optimum dose and dosing schedule. Cell therapies used in combination with the interferon analog can be administered to the animal by the optimum route determined for the cell therapy. One preferred route of administration for cell therapies is intravenous infusion.

In the method of the present invention, compositions can be administered to any animal and preferably, to any member of the Vertebrate class, Mammalia, including, without limitation, primates, rodents, livestock and domestic pets. Livestock include mammals to be consumed or that produce useful products (e.g., sheep for wool production). Preferred mammals to protect include humans, dogs, cats, mice, rats, sheep, cattle, horses and pigs, with humans being particularly preferred.

One embodiment of the invention is a method for stimulating expression of MHC Class II proteins in cells of an animal, comprising administering one or more effective doses of an interferon gamma analog to the animal.

Another embodiment of the invention is a method for stimulating expression of MHC Class I proteins in cells of an animal, comprising administering one or more effective doses of an interferon gamma analog to the animal.

Another embodiment of the invention is a method for stimulating PD-L1 expression in cells of an animal, comprising administering one or more effective doses of an interferon gamma analog to the animal.

Another aspect of the invention is a method for stimulating expression of MHC Class II proteins in tumor cells in an animal, comprising administering one or more effective doses of an interferon gamma analog to the animal with a tumor.

Another aspect of the invention is a method for stimulating expression of MHC Class I proteins in tumor cells in an animal, comprising administering one or more effective doses of an interferon gamma analog to the animal with a tumor.

Another aspect of the invention is a method for stimulating PD-L1 expression in tumor cells in an animal, comprising administering one or more effective doses of interferon gamma analog to the animal with a tumor.

Another aspect of the invention is a method for stimulating expression of antigen processing proteins and antigen presentation proteins in cells in an animal comprising administering one or more effective doses of an interferon gamma analog to the animal. Examples of antigen processing and antigen presentation proteins, in addition to MHC-I and MHC-II, include but are not limited to IRF1, STAT1, NLRC5, proteasomal subunits (i.e., LMP2, LMP7 and LMP10), peptidases (ERAP1, ERAP2, etc.), TAP and tapasin.

Another aspect of the invention is a method for stimulating expression of antigen processing and antigen presentation proteins in tumor cells in an animal comprising administering one or more effective doses of an interferon gamma analog to the animal.

Another embodiment of the invention is a method for inhibiting growth of a tumor in an animal comprising administering to the animal with a tumor one or more effective doses of an interferon gamma analog, wherein the interferon gamma analog increases tumor cell MHC-II protein expression. Optionally, the animal may be administered one or more effective doses of an anti-PD-L1 antibody or an anti-PD-1 antibody, another checkpoint inhibitor antibody, or a T cell agonist drug.

Another embodiment of the invention is a method for inhibiting growth of a tumor in an animal comprising administering to the animal with a tumor one or more effective doses of an interferon gamma analog, wherein the interferon gamma analog increases tumor cell MHC-I protein expression. Optionally, the animal may be administered one or more effective doses of an anti-PD-L1 antibody or an anti-PD-1 antibody, another checkpoint inhibitor antibody, or a T cell agonist drug.

Another embodiment of the invention is a method for inhibiting growth of a tumor in an animal comprising administering to the animal with a tumor one or more effective doses of an interferon gamma analog, wherein the interferon gamma analog increases tumor cell PD-L1 protein expression. Optionally, the animal may be administered one or more effective doses of an anti-PD-L1 antibody or an anti-PD-1 antibody, another checkpoint inhibitor antibody, or a T cell agonist drug.

Another embodiment of the invention is a method for inhibiting growth of a tumor in an animal comprising administering to the animal with a tumor one or more effective doses of an interferon gamma analog and one or more effective doses of an anti-PD-L1 antibody or an anti-PD-1 antibody, wherein the interferon gamma analog induces expression of PD-L1 in the tumor cell, and wherein tumor growth inhibition with the combination of the interferon gamma analog and the anti-PD-L-1 antibody or the anti-PD-1 antibody is greater than the tumor growth inhibition obtained by treatment with the interferon gamma analog alone or the anti-PD-L1 antibody alone or the anti-PD-1 antibody alone.

Another aspect is a method for stimulating an anti-tumor immune response in an animal with a tumor comprising administering to the animal with a tumor one or more effective doses of an interferon gamma analog and one or more effective doses of an anti-PD-L1 antibody or an anti-PD-1 antibody, wherein the interferon gamma analog induces expression of PD-L1 in the animal's cell, and wherein the anti-tumor immune response obtained with the combination of the interferon gamma analog and the anti-PD-L-1 antibody or anti-PD-1 antibody is greater than the anti-tumor immune response obtained by treatment with the interferon gamma analog alone or the anti-PD-L1 antibody alone or the anti-PD-1 antibody alone.

Another aspect is a method for stimulating an anti-tumor immune response in an animal with a tumor comprising administering to the animal one or more effective doses of an interferon gamma analog, wherein the interferon gamma analog increases expression of PD-L1 in the tumor cell. Optionally, the animal may be administered one or more effective doses of an anti-PD-L1 antibody, an anti-PD-1 antibody, another checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-cytotoxic T-lymphocyte Antigen 4 (anti-CTLA4) antibody, an anti-lymphocyte-activator gene 3 (anti-LAG-3) antibody, an anti-T-cell immunoglobulin mucin-3 antibody (anti-TIM 3 antibody), an anti-tissue inhibitor of matrix metalloproteinase antibody (anti-TIMP antibody), and an anti-T-cell immunoreceptor with Ig and ITIM domains antibody (anti-TIGIT antibody). Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist (also known as tumor necrosis factor ligand superfamily member 9), and a CD40 agonist. Preferably the T cell agonist protein is an antibody.

Another aspect is a method for stimulating an anti-tumor immune response in an animal with a tumor, comprising administering to the animal with a tumor one or more effective doses of an interferon gamma analog, wherein the interferon gamma analog increases expression of MHC Class II proteins in the tumor cell. Optionally, the animal may be administered one or more effective doses of an anti-PD-L1 antibody, an anti-PD-1 antibody, another checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-LAG-3 antibody, an anti-TIM 3 antibody, an anti-TIMP antibody, and an anti-TIGIT antibody. Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist, and a CD40 agonist. Preferably the T cell agonist protein is an antibody.

Another aspect is a method for stimulating an anti-tumor immune response in an animal with a tumor, comprising administering to the animal with a tumor one or more effective doses of an interferon gamma analog, wherein the interferon gamma analog increases expression of MHC Class I proteins in the tumor cell. Optionally, the animal may be administered one or more effective doses of an anti-PD-L1 antibody, an anti-PD-1 antibody, another checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-LAG-3 antibody, an anti-TIM 3 antibody, an anti-TIMP antibody, and an anti-TIGIT antibody. Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist, and a CD40 agonist. Preferably the T cell agonist protein is an antibody.

Another aspect of the invention is a method for increasing the number of natural killer (NK) cells in an animal, comprising administering to the animal one or more effective doses of an interferon gamma analog.

Another aspect of the invention is a method for stimulating a graft versus leukemia anti-tumor immune response in an animal comprising administering to an animal with a leukemia an interferon gamma analog. Preferably, the animal with a leukemia is administered both the interferon gamma analog as well as immune cells from another animal (allogeneic donor immune cells). Preferably, the allogeneic donor immune cells include allogeneic donor T lymphocytes (also referred to as allogeneic T cells). Optionally, the animal may also be administered one or more effective doses of an anti-PD-L1 therapy or an anti-PD-1 therapy. Optionally, the animal may also be administered one or more effective doses of an anti-PD-L1 antibody, an anti-PD-1 antibody, another checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-LAG-3 antibody, an anti-TIM 3 antibody, an anti-TIMP antibody, and an anti-TIGIT antibody. Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist, and a CD40 agonist. Preferably the T cell agonist protein is an antibody.

Another aspect is a method for stimulating a graft versus leukemia anti-tumor immune response in an animal comprising administering to an animal with a leukemia an interferon gamma analog that stimulates increased expression of MHC-II proteins in the leukemia cells. Preferably, the animal with a leukemia is administered both the interferon gamma analog as well as immune cells from another animal (allogeneic donor immune cells). Preferably, the allogeneic donor immune cells include allogeneic donor T lymphocytes (also referred to as allogeneic T cells). Optionally, the animal may also be administered one or more effective doses of an anti-PD-L1 therapy or an anti-PD-1 therapy. Optionally, the animal may be administered one or more effective doses of an anti-PD-L1 antibody, an anti-PD-1 antibody, another checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-LAG-3 antibody, an anti-TIM 3 antibody, an anti-TIMP antibody, and an anti-TIGIT antibody. Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist, and a CD40 agonist. Preferably the T cell agonist protein is an antibody.

Another aspect is a method for stimulating a graft versus leukemia anti-tumor immune response in an animal comprising administering to an animal with a leukemia an interferon gamma analog that stimulates increased expression of MHC-I proteins in the leukemia cells. Preferably, the animal with a leukemia is administered both the interferon gamma analog as well as immune cells from another animal (allogeneic donor immune cells). Preferably, the allogeneic donor immune cells include allogeneic donor T lymphocytes (also referred to as allogeneic T cells). Optionally, the animal may also be administered one or more effective doses of an anti-PD-L1 therapy or an anti-PD-1 therapy. Optionally, the animal may be administered one or more effective doses of an anti-PD-L1 antibody, an anti-PD-1 antibody, another checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-LAG-3 antibody, an anti-TIM 3 antibody, an anti-TIMP antibody, and an anti-TIGIT antibody. Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist, and a CD40 agonist. Preferably the T cell agonist protein is an antibody.

Another aspect is a method for stimulating a graft versus leukemia anti-tumor immune response in an animal comprising modifying the animal's leukemia cells with a gene encoding class II transactivator (CIITA). Preferably, the leukemia cells modified with the gene encoding CIITA express increased levels of MHC-II proteins and/or other proteins involved in antigen processing and antigen presentation. Optionally, the animal may be administered one or more effective doses of an anti-PD-L1 antibody, an anti-PD-1 antibody, another checkpoint inhibitor antibody, or a T cell agonist protein. Preferably the checkpoint inhibitor antibody is selected from the group consisting of, but not limited to an anti-PD-L1 antibody, an anti-PD-1 antibody, an anti-LAG-3 antibody, an anti-TIM 3 antibody, an anti-TIMP antibody, and an anti-TIGIT antibody. Most preferably, the checkpoint inhibitor antibody is an anti-PD-L1 antibody or an anti-PD-1 antibody. Preferably, the T cell agonist drug is a protein drug selected from the group consisting of an OX-40 agonist, a 4-1BB agonist, and a CD40 agonist. Preferably the T cell agonist protein is an antibody.

The methods described herein are generally applicable to all types of tumors and cancer types, including but not limited to leukemias, including acute myelogenous leukemias (AML, also referred to as acute myeloid leukemia), lymphomas, myelomas, glioblastomas, melanomas, lung cancers, kidney cancers, liver cancers, pancreatic cancers, breast cancers, triple negative breast cancers, colon cancers, bladder cancers, fibrosaracoma cancers, gastric cancers, esophageal cancers, prostate cancers, and bone cancers. The methods may be particularly useful for treating tumor cells and cancer cells that express low or no levels of MHC-I, MHC-II, or PD-L1 proteins. One preferred tumor cell type is an AML tumor that has relapsed after HCT. More preferably, the tumor type is an AML tumor that has relapsed after HCT and expresses low or no levels of one or more of the following proteins: MHC-I proteins, MHC-II proteins, or PD-L1 proteins. Most preferably, the relapsed AML tumor cells express lower levels of one or more proteins selected from the group consisting of MHC-I proteins, MHC-II proteins, or PD-L1 proteins compared to the patient's AML cells prior to relapse. Another preferred tumor type is a breast cancer tumor, more preferably a triple negative breast cancer tumor, and most preferably a triple negative breast cancer tumor that expresses low or no levels of one or more of the proteins selected from the group consisting of MHC-I proteins, MHC-II proteins, or PD-L1 proteins.

The methods described herein will also be useful for treating tumors and cancers in an animal in combination with other chemotherapeutic agents, anti-cancer drugs, checkpoint inhibitor antibodies, particularly checkpoint inhibitor monoclonal antibodies, cell therapies such as donor immune cells, donor lymphocytes, donor T lymphocytes, donor white blood cells, donor macrophages, donor NK cells, donor B cell lymphocytes, donor dendritic cells, and engineered anti-tumor T cell therapies, engineered anti-tumor macrophage cell therapies, anti-tumor NK cell therapies, anti-tumor B cell therapies, and anti-tumor dendritic cell therapies.

As demonstrated in the Examples below, a long-acting interferon gamma analog was engineered and shown to increase expression of MHC-I proteins, MHC-II proteins, and PD-L1 proteins in tumor cells in vitro and in an animal. A non-long-acting interferon gamma analog also was shown to increase expression of MHC-I proteins, MHC-II proteins, and PD-L1 proteins in tumor cells in vitro and in an animal. However, the inventors discovered that treatment of an animal containing a tumor with the long-acting interferon gamma analog increased tumor cell expression of MHC-I proteins, MHC-II proteins, and PD-L1 proteins to a greater extent than treating an animal with a non-long-acting interferon gamma analog. In addition, the long-acting interferon gamma analog was shown to increase expression of MHC-I proteins, MHC-II proteins, and PD-L1 proteins in cells of an animal following administration of the long-acting interferon gamma analog to the animal. In addition, the long-acting interferon gamma analog was shown to re-sensitize an allogeneic T cell resistant tumor to killing by allogeneic donor lymphocytes (allogeneic donor T cells) and extend survival in mice. The results establish simple, translatable strategies to enhance the immunogenicity of tumors in an animal and to inhibit their growth and extend survival using an interferon gamma analog alone or in combination with other anti-cancer drugs such as checkpoint inhibitor antibodies, T cell agonist drugs, chemotherapeutic agents, cell therapies including but not limited to donor lymphocytes, donor T lymphocytes, donor white blood cells, donor macrophages, donor NK cells, donor B cell lymphocytes, donor dendritic cells, engineered T cell therapies, engineered macrophage cell therapies, engineered NK cell therapies, engineered B cell therapies, or other cancer drugs.

The following experimental results are provided for purposes of illustration and are not intended to limit the scope of the invention.

### EXAMPLES

Example 1. Construction of a gene encoding the human IFNg (Q67C/Q1 deletion) analog (also referred to as IFNg (Q67C) or PEG hIFNg, methods for its expression in E. coli, purification, modification with a 20 kDa linear maleimide polyethylene glycol (PEG), and purification of the PEGylated IFNg (Q67C) protein are described in U.S. Pat. Nos. 9,296,804, 8,618,256, 8,617,531, 7,964,184 and 7,959,909 (each are herein incorporated by reference) for other PEG hIFNg cysteine muteins such as the IFNg (L103C mutein). The methods are also essentially similar to those described in Fam et al (2014) Journal of Interferon and Cytokine Research 34:759-768, for preparation of the PEG hIFNg (L103C) protein.

The PEG hIFNg (Q67C) protein has also been prepared using protein obtained following E. coli fermentation. One E. coli cell pellet from a 10 L fermentation was thawed and resuspended in 400 mL of cold 20 mM Tris, 1 mM EDTA, pH 8.0 buffer with an immersion blender or polytron mixer, and then passed 3 times through a NIRO cell homogenizer set to 700 bar. The lysed cells were centrifuged and the insoluble pellet fraction (referred to as inclusion bodies) containing the majority of the insoluble hIFNg protein was stored frozen at −20° C. The inclusion bodies were solubilized for 2 h by stirring in 20 mM Tris, 6M guanidine, 10 mM TCEP, pH 7.5, and then clarified by centrifugation at 8,000 rpm for 45 min. The supernatant was isolated and diluted about 12 fold into cold 20 mM Tris, 1 mM TCEP, 1 mM EDTA, pH 7.5 and refolded overnight at 4° C. The next day the refold solution was centrifuged at 8,000 rpm for 45 min and the soluble supernatant fraction was diluted 1/1.5 with Buffer A (20 mM HEPES, 1 mM TCEP, pH 7.5) and applied to an S-Sepharose Fast Flow column equilibrated in Buffer A. After washing with 38% Buffer B (20 mM HEPES, 1 mM TCEP, 1M NaCl, pH 7.5), the bound proteins were eluted with a 30 column volume gradient of 38% to 66% Buffer B. Fractions enriched in the IFNg protein were identified by SDS-PAGE, pooled and quantitated by A280 using an extinction coefficient of 0.689. The S-Fast Flow pool was further purified on Phenyl-High Performance column. The S column pool was diluted ½ in 20 mM MES, 3M (NH4)2SO4, 0.05 mM TCEP pH 6.7 (Dilution Buffer) and applied to the Phenyl-High Performance column equilibrated with Buffer A (20 mM MES, 1.5M (NH4) 2SO4, 0.05 mM TCEP pH 6.7). The column was washed with Buffer A, and bound proteins eluted using a 10 column volume step gradient of 40% Buffer B (20 mM MES, 0.05 mM TCEP, 25% propylene glycol pH 6.7). Column fractions were analyzed by SDS-PAGE. Fractions containing hIFNg protein were pooled, quantitated by A280 and immediately PEGylated. The Phenyl pool was diluted ½ with MilliQ water and PEGylated with a 1:1 molar ratio of TCEP to protein and a 3 fold molar ratio of 20 kDa-maleimide PEG (NOF ME-200MAOB) to the IFNg protein pool. The reaction was allowed to proceed for 1 h at room temperature, then moved to 4° C. for overnight. The following morning the reaction was further diluted ⅓ in a 20 mM succinic acid, pH 6.0 buffer (BufferA) and loaded onto an S-Sepharose High Performance column to separate the PEGylated protein from the unreacted protein and unreacted PEG. After loading, the column was washed with 30% Buffer B (20 mM succinic acid, 1M NaCl, pH 6.0), followed by elution with a 25 column volume 30-55% Buffer B gradient. Column fractions were analyzed by SDS-PAGE, and fractions containing the 20 kDa PEG IFNg protein pooled and stored frozen at −70° C. Since hIFNg is a homodimeric protein, fractions were chosen that were enriched for the diPEGylated homodimer, i.e., fractions in which both monomers of the homodimer were PEGylated).

### Example 2. Cloning, Expression and Purification of Mouse IFNg and PEGylated Mouse IFNg Cysteine Analogs

A murine IFNg (mIFNg) homolog of the human IFNg (Q67C) protein was constructed for testing in rodent animal models. A gene encoding mIFNg (SEQ ID NO: 2) with an added N-terminal methione residue was synthesized and cloned into plasmid pUC57. The gene included extra DNA at each end to encode restriction enzyme sites for cloning purposes. The wild type (WT) mIFNg protein contains a C-terminal cysteine residue (amino acid 133; referred to as C133) not present in the human IFNg protein. Polymerase chain reaction (PCR) mutagenesis was used to change the C133 residue in to a serine (referred to as C133S), to ensure that the cysteine added later (corresponding to Q67C in hIFNg) was the only unpaired cysteine residue in the protein. The mIFNg (C133S) gene was subcloned into the pET21 (a) expression vector under control of the T7 phage promoter and used to transform E. coli strain BL21 (DE3). The muIFNg (C133S) protein protein was expressed, refolded and purified using the following procedures.

For expression studies, the E. coli strain was grown overnight in LB media supplemented with 100 μg/mL ampicillin and 1% glucose (w/v) at 37° C. in an air shaker set to 235 rpm. The next morning, 4 mL of the overnight culture was diluted into 400 mL LB media containing 100 μg/mL ampicillin in a 2 L flask and the cells shaken at 235 rpm at 37° C. When the cells reached an optical density at 600 nm of 0.5 to 0.6, IPTG (isopropyl beta-D-1 thioglalactopyranoside) was added to the culture to a final concentration of 0.5 mM and the temperature was increased to 42° C. to induce expression of the IFNg protein. After overnight growth the induced cells were harvested by centrifugation and stored frozen at −20° C. The cell pellets from 4 flasks were combined, suspended by polytron in 20 mM Tris, 1 mM EDTA, pH 8.0 buffer, and then passed 3 times through a NIRO cell homogenizer set to 700 BAR. The lysed cells were centrifuged and the insoluble pellet fraction (referred to as inclusion bodies) containing the majority of the insoluble IFNg protein was stored frozen at −20° C. The inclusion bodies were solubilized for 2 h by stirring in 20 mM Tris, 6M guanidine, pH 8, then insolubles removed by centrifugation at 8,000 rpm for 20 min. The resulting supernatant was isolated and diluted about 28 fold into 20 mM Tris, 0.3M guanidine, 1 mM EDTA, pH 8.0 and refolded overnight at 4° C. The next day the refold solution was centrifuged at 8,000 rpm for 45 min and the supernatant fraction isolated and diluted 1:1 with Buffer A (20 mM Tris pH 7.0) and applied to an S-Sepharose high performance column equilibrated in Buffer A. After washing with Buffer A, the bound proteins were eluted with a 20 column volume gradient of 25% to 75% Buffer B (20 mM Tris, 1M NaCl, pH 7). Fractions enriched in the IFNg protein were identified by SDS-PAGE, pooled, diluted 10× in Buffer A (20 mM Tris pH 8.0) and applied to sequential, tandemly linked Q-sepharose, S-sepharose columns using Buffer A. The mIFNg protein flows through the Q column and is bound by the S-column. The Q-column removes endotoxin from the protein as the protein flows through the column. Once the mIFNg protein had passed through the Q column, the Q column was detached from the S-column, and bound proteins eluted from the S-column using a 20 column volume gradient of 25% to 75% Buffer B (20 mM Tris, 1M NaCl, pH 8). Column fractions were analyzed by SDS-PAGE and fractions enriched in the IFNg protein were pooled and stored frozen at −20° C. to −70° C. The approximate salt concentration of the pooled IFNg protein was 350 mM sodium chloride in is 20 mM Tris, pH 8.

PCR mutagenesis was used to create several cysteine muteins in the mIFNg (C133S) protein. The muteins were intended to be murine versions of the hIFNg (Q67C) mutein. Human and mouse IFNg are not aligned perfectly in this region. Based upon a best alignment of the amino acid sequences serine-66 was changed to a cysteine [referred to as mIFNg (S66C/C133S) or simply mIFNg (S66C)]. The mIFNg (Q63C/C133S) and mIFNg (A64C/C133S) muteins were also constructed.

The mIFNg (S66C/C133S) protein was expressed and purified as described for the mIFNg (C133S) protein above through the inclusion body isolation steps. The inclusion bodies for the mIFNg (S66C/C133S) protein were solubilized 1-2 h by stirring in 20 mM Tris, 6M guanidine, 10 mM TCEP [Tris(carboxyethyl) phosphine], pH 8, and then insolubles removed by centrifugation at 8,000 rpm for 20 min. The supernatant fraction was isolated and diluted about 28-fold into 20 mM Tris, 0.3M Guanidine, 1 mM TCEP, 1 mM EDTA, pH 8.0 and refolded overnight at 4° C. The next day the refold solution was centrifuged at 8,000 rpm for 45 min and the soluble supernatant fraction isolated and diluted 1:1 with Buffer A (20 mM Tris, ImM TCEP, pH 7.0) and applied to an S-Sepharose High performance column equilibrated in Buffer A. After washing with Buffer A, the bound proteins were eluted with a 20 column volume gradient of 25% to 75% Buffer B (20 mM Tris, 1 mM TCEP, 1M NaCl, pH 7). Fractions enriched in the IFNg (S66C/C133S) protein were identified by SDS-PAGE, pooled, diluted 10× in Buffer A (20 mM Tris, 0.05 mM TCEP pH 7.2) and applied to sequential, tandemly-linked Q-sepharose, S-sepharose columns using Buffer A. The mIFNg (S66C/C133S) protein flows through the Q column and is bound by the S-column. The Q-column removes endotoxin from the protein as the protein flows through the column. Once the mIFNg (S66C/C133S) protein had passed through the Q column, the Q column was detached from the S-column, the S-column washed with 25% Buffer B and bound proteins eluted from the S-column using a 10 column volume step gradient of 100% Buffer B (20 mM Tris, 0.05 mM TCEP 1M NaCl, pH 7.2). Column fractions were analyzed by SDS-PAGE, fractions enriched in the mFNg (S66C/C133S) protein were pooled and stored overnight at 4° C. The following morning the pool was warmed to room temperature and PEGylation of the mIFNg (S66C/C133S) protein was accomplished by adding a 1 molar ratio of TCEP and a 3 fold molar ratio of a linear 20 kDa-maleimide PEG (NOF ME-200MAOB) to the mIFNg (S66C/C133S) protein pool. The reaction was allowed to proceed for 2 h at room temperature. The reaction was diluted 5-fold in a 20 mM Tris, pH 7.2 buffer (Buffer A) and loaded onto a final S-sepharose High performance column to separate the PEGylated protein from the unreacted protein and unreacted PEG. After loading, the column was washed with 15% Buffer B (20 mM Tris, 1M NaCl, pH 7.2), followed by elution of the proteins with a 30 column volume 15-50% Buffer B gradient. Column fractions were analyzed by SDS-PAGE, fractions containing the 20 kDa PEG mIFNg (S66C/C133S) protein and stored frozen at −70° C. Since mIFNg is a homodimeric protein, fractions were chosen that were enriched for the diPEGylated homodimer, i.e., fractions in which both monomers of the homodimer were PEGylated). The approximate salt concentration of the pooled PEG mIFNg (S66C/C133S) protein was 350 mM sodium chloride in 20 mM Tris, pH 7.2. In some experiments the buffer for the final S-column was pH 6.8.

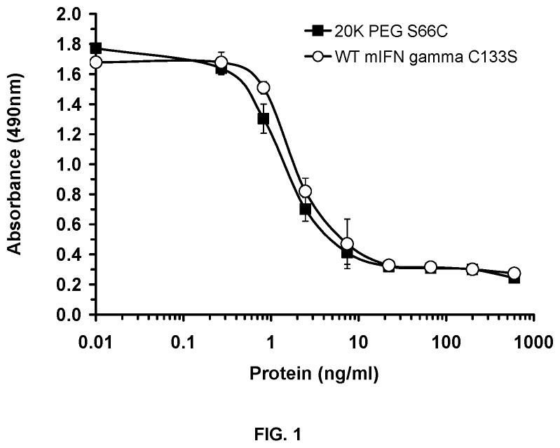

In vitro bioactivities of the purified proteins were measured in a cell growth inhibition assay using the murine B16BL6 melanoma cell line (obtained from National Cancer Institute). Methods are essentially similar to those described for hIFNg in U.S. Pat. Nos. 9,296,804, 8,618,256, 8,617,531, 7,964,184 and 7,959,909 (each are herein incorporated by reference), and Fam et al (2014) Journal of Interferon and Cytokine Research 34:759-768, except for the different cell line. Dose response curves for the PEG-mIFNg (S66C/C133S) protein and the WT mIFNg (C133S) protein are shown in FIG. 1. The unPEGylated mIFNg (S66C/C133S) protein displayed similar tumor cell growth inhibition curves as mIFNg (C133S) and the PEG-mIFNg (S66C/C133C) protein. All three proteins had similar IC₅₀s (protein concentration resulting in half-maximal growth inhibition) of ˜5 ng/ml in this bioassay, indicating minimal, if any loss of biological activity as a result of PEGylation of the added cysteine residue. The optimized assay conditions used 10,000 B16 cells per well of a 96 well tissue culture plate, and the cells are incubated for 4 days at 37° C. Proteins were assayed using triplicate wells for each protein concentration, and the results for the triplicate wells were averaged. Cell number was measured by adding the MTS dye to the test wells on day 4. Absorbance of the wells, which is proportional to cell number, as read about 1 hour later at 490 nm using a plate reader.

### Example 3. Single Injection Safety Toxicology Study of mIFNg and PEG-mIFNg (S66C) in Rats

A safety toxicology study was performed in rats to identify safe doses of the PEG-mIFNg (S66C) protein for use in rodents. Murine IFNg is active in rats. Rats (albino adult Sprague-Dawley rats, weighing ˜200 g) received a single subcutaneous (sc) injection of WT mIFNg (C133S) and three different doses of PEG-mIFNg (S66C) and followed for 14 days for signs of toxicity. The dose of WT mIFNg used in the toxicology study, 100 μg/kg, is the same dose used by others in murine p47 phox efficacy studies (Jackson et al., 2001). Different groups of rats received 30, 100 or 300 μg/kg of the PEG-mIFNg (S66C/C133S) protein, which correspond to human equivalent doses of about 4.8, 16, and 48 μg/kg, adjusting for body mass differences between rats and humans. Only the protein portion of the PEG IFNg protein was used for dose calculations. For comparison, the typical dose of a non-long-acting hIFNg analog (Actimmune) used in humans about 1.5 μg/kg administered 3×/week by subcutaneous injection (weekly dose of about 4.5 μg/kg. A fifth group of rats received vehicle solution (phosphate buffered saline, PBS). Each test group comprised 6 rats (3 males, 3 females). Rats received a gross necropsy at time of sacrifice on day 15. On day 0 prior to dosing and on Days 4 and 15 one mL blood samples were obtained. A portion of the blood samples was collected in EDTA coagulant tubes and used for a complete blood cell count (CBC analysis). The remaining portion (0.3 mL) was used to prepare serum for serum chemistry analyses, which tested for ˜ 20 standard blood chemistry parameters, including total protein, albumin, globulin, albumin/globulin ratio, sodium, potassium, chloride, calcium, phosphorous, alanine aminotransferase, aspartate aminotransferase, lactate dehydrogenase, alkaline phosphatase, creatine kinase, amylase, glucose, urea nitrogen, creatinine, cholesterol (total), triglycerides, bilirubin (total), and uric acid.

No toxicities were noted during the live phase of the study and no obvious tissue toxicities were noted at necropsy. The blood chemistries and CBC data showed no significant differences between animals receiving vehicle solution (PBS), WT mIFNg (C133S) or any dose of PEG-mIFNg (S66C/C133S) protein. The results from these studies show that single doses of up to 300 μg/kg of PEG-mIFNg (S66C/C133S) are safe in rats.

### Example 4. Pharmacokinetic (PK) and Safety Study of WT mIFNg and PEG mIFNg (S66C) in Mice

Balb/c mice were administered a single subcutaneous (sc) injection of 0.5 mg/kg wild type (WT) mIFNg or 0.05, 0.15, or 0.5 mg/kg PEG mIFNg, which corresponds to human equivalent doses of about 4, 12.1, and 40 μg/kg, respectively, adjusting for body mass differences between mice and humans. Mice were followed for 7 days. No safety issues were observed. At pre-dose, 2 h, 4 h, 10 h, 24 h, 48 h, 72 h, 96 h, 120 h, and 168 h, four mice per group (2M/2F) were sacrificed and plasma collected in sodium EDTA tubes. The 2 h time point was not collected for mice administered PEG mIFNg and the 120 h and 168 h time points were not collected for mice administered mIFNg. Plasma levels of WT mIFNg and PEG mIFNg were measured using mIFNg QUANtiKINE™ ELISA kits obtained from R&D Systems (Catalog number MIF00). Serial dilutions of the mIFNg standards included with the ELISA kit were used for measuring WT mIFNg plasma samples. Serial dilutions of PEG mIFNg were used as the standards for measuring PEG mIFNg plasma samples. Plasma samples were initially diluted 10-fold into RPMI media containing 10% fetal bovine serum and then serially in the same media. Pharmacokinetic parameters were calculated using PK Solver software. Plasma levels of the proteins (means+/−standard deviations) are listed in Table 1. The limit of detection of mIFNg in the ELISA was 9.4 pg/mL (94 pg/mL in plasma when adjusted for the initial 10-fold plasma dilution). The limit of detection of PEG mIFNg in the ELISA was 50 μg/mL (500 μg/mL in plasma when adjusted for the initial 10-fold plasma dilution).

TABLE 1
Plasma levels of WT mIFNg and PEG mIFNg following
subcutaneous administration to mice.

| Hour | mIFNg | PEG mIFNg | PEG mIFNg | PEG mIFNg |
|---|---|---|---|---|
| post | 0.5 mg/kg | 0.5 mg/kg | 0.15 mg/kg | 0.05 mg/kg |
| dosing | (ng/mL) | (ng/ml) | (ng/ml) | (ng/ml) |
| 0 | 0 +/− 0 | 0 +/− 0 | 0 +/− 0 | 0 +/− 0 |
| 2 | 37.9 +/− 8 | N/A | N/A | N/A |
| 4 | 6.1 +/− 3 | 500.1 +/− 264 | 82.0 +/− 17 | 7.7 +/− 4 |
| 10 | 0 +/− 0 | 509.1 +/− 56 | 54.0 +/− 12 | 7.5 +/− 3 |
| 24 | 0 +/− 0 | 243.0 +/− 25 | 25.3 +/− 11 | 3.9 +/− 1 |
| 48 | 0 +/− 0 | 57.2 +/− 9 | 6.0 +/− 1 | 1.0 +/− 0 |
| 72 | 0 +/− 0 | 12.8 +/− 2 | 2.0 +/− 0 | 0 +/− 0 |
| 96 | 0 +/− 0 | 2.7 +/− 1 | 0.5 +/− 0.5 | 0 +/− 0 |
| 120 | N/A | 1.3 +/− 0 | 0 +/− 0 | 0 +/− 0 |
| 168 | N/A | 0 +/− 0 | 0 +/− 0 | 0 +/− 0 |

N/A not applicable. Time point not collected.
N = 4 mice (2M/2F) per time point per group. Data are means +/− standard deviations.

The data show (see Table 2) that a single sc dose of mIFNg reaches peak plasma levels 2 h after dosing and is undetectable (<94 μg/mL) in plasma 10 h after dosing. The protein's half-life is about 0.76 h. By contrast, PEG mIFNg reaches peak plasma levels between 4 to 10 h after dosing and is cleared more slowly, with a terminal half-life of about 12 h. Peak plasma levels of the 0.5, 0.15, and 0.05 mg/kg PEG mIFNg doses were 509.1, 82.0 and 7.7 ng/mL, respectively. PEG mIFNg (0.5 mg/kg dose) maintains a plasma level greater than 10 ng/mL for 72 h and a plasma level greater than 1 ng/ml for 120 h. PEG mIFNg (0.15 mg/kg dose) maintains a plasma level greater than 10 ng/mL for 24 h, a plasma level greater than 5 ng/mL for 48 h, a plasma level greater than 1 ng/ml for 72 h, and a plasma level of 0.5 ng/ml or higher for 96 h. PEG mIFNg (0.05 mg/kg dose) maintains a plasma level greater than 5 ng/ml for 10 h and a plasma level of 1 ng/ml or higher for 48 h. Systemic exposure, as measured by area under the protein concentration time curve (AUC0-t) were higher for all doses of PEG mIFNg compared to mIFNg.

TABLE 2
Subcutaneous (sc) pharmacokinetic (PK)
properties of mIFNg and PEG mIFNg

| | | Tmax | Cmax | AUC0-t | T½ |
|---|---|---|---|---|---|
| Protein | Dose | (h) | (ng/ml) | ng/ml*h | (h) |
| mIFNG | 0.5 mg/kg | 2 | 37.9 | 75.9 | 0.76 |
| PEG- mIFNG | 0.5 mg/kg | 10 | 509.1 | 13,969 | 12.2 |
| PEG- mFNG | 0.15 mg/kg | 4 | 82 | 1,599 | 12.6 |
| PEG- mIFNG | 0.05 mg/kg | 4 | 7.7 | 200 | 13.0 |

### Example 5. Reversal of MHC-II Downregulation Enhances Graft-Versus-Leukemia (GvL) Anti-Tumor Effects

Relapse after allogeneic hematopoietic stem cell transplantation (HCT) represents the most common type of treatment failure and is thought to result in part from AML cell escape from immune pressure exerted by donor immune cells (the “Graft-versus-leukemia” effect, GvL). It has been previously reported downregulation of MHC-II expression in up to half of AML cases relapsing after transplantation, but it is unclear whether a) loss of MHC-II contributes mechanistically to loss of GvL, and b) whether reversal of MHC-II downregulation can restore GvL. The role of MHC-II in mediating GvL in a mouse model of minor-antigen mismatch HCT was tested where C57BL/6 mice (B6) bearing syngeneic AML cells (H-2b) were co-transplanted with or without CD3+ T cells from C3.SW-H2b/SnJ mice (H-2b). Since murine AML cells do not generally express MHC-II at baseline, MLL-AF9-driven B6 AML cells were transduced with a fluorescently-tagged retrovirus expressing CIITA, the main transcriptional regulator of MHC-II genes. The inventors discovered that MHC-II-expressing AML cells were selectively deleted when co-transplanted with donor T cells, in contrast to animals that received no T cells or mice that received AML cells transduced with empty vector. To extend these findings to human cells, both MHC-II blocking antibodies and CRISPR-mediated disruption of CIITA in human AML cell lines and primary samples were used. Loss or blocking of MHC-II led to diminished ability of AML cells to stimulate MHC-mismatched donor T cells in a mixed lymphocyte reaction.

Loss of MHC-II expression in AML cells can be reversed in vitro by IFNg. The ability of hIFNg to enhance graft versus leukemia (GvL) immune responses was tested by culturing AML cell lines and primary patient AML tumor cells with or without hIFNg for 48 hours in vitro. hIFNg treatment enhanced the ability of AML cells from some samples to stimulate third-party T cells in a mixed lymphocyte reaction. Enhanced stimulation was observed in both CD4 and CD8 cell classes, suggesting that hIFNg may potentiate GvL through MHC-II-dependent and MHC-II-independent pathways.

### Example 6. PEG IFNg Increases Expression of MHC-I and MHC-II Proteins on Human AML Cells in Mice and Increases Expression of the Proteins to a Greater Extent than IFNg

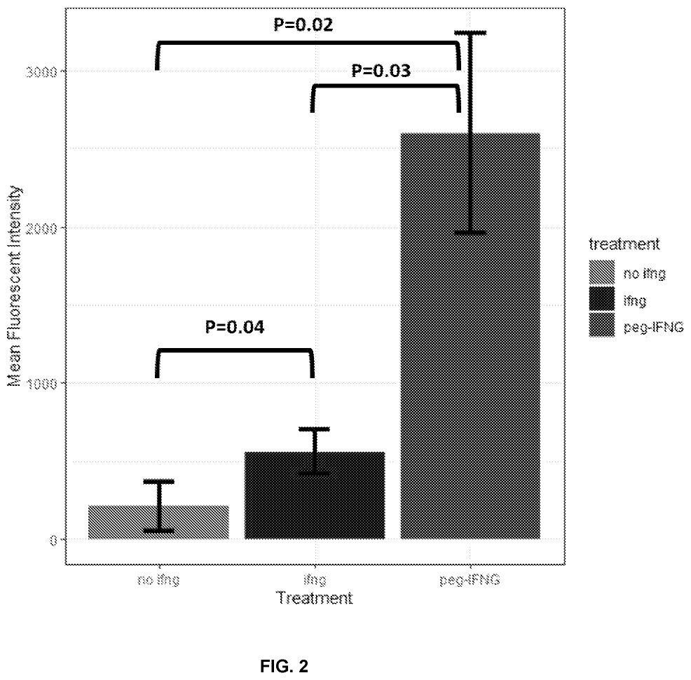

Since the use of IFNg in vivo may be limited by its short half-life, we tested the long-acting PEG-hIFNg (Q67C) protein (see Example 1) in a xenograft model of AML. Immunodeficient NSG-SGM3 mice (NOD.Cg-Prkdcscid Il2rgtm1Wj1 Tg (CMV-IL3,CSF2,KITLG) 1Eav/MloySzJ, Jackson Labs) were engrafted with MHC-II-low expressing human AML cells from a patient sample. After engraftment, mice (n=3 per each group) received six doses of vehicle (phosphate-buffered saline), hIFNg (from Peprotech), or PEG-hIFNg by sc injection 3×/week on Monday, Wednesday and Friday. hIFNg and PEG hIFNg were administered at a dose of 15 micrograms (mcg) per mouse per injection (about 0.75 mg/kg/injection, which corresponds to a human equivalent dose of about 60 mcg/kg/injection). After treatment, mice were sacrificed and MHC-II protein expression was quantified on the human AML cells isolated from bone marrow of treated mice by flow cytometry. PEG-hIFNg was well tolerated with no effect on overall survival or body weight. As shown in FIG. 2, IFNg treatment increased MHC-II expression on the AML cells modestly compared to vehicle (2.7-fold, p=0.04), while PEG-hIFNg increased MHC-II expression to a much greater extent on the AML cells compared to vehicle (12-fold, p=0.02) or compared to hIFNg (about 4.4-fold, p=0.03). Together, these findings indicate that IFN analogs, and especially long-acting IFNg analogs such as PEG-IFNg, will increase tumor cell MHC II expression and effectively enhance GvL in AML patients after HCT, particularly in patients with low MHC-II-expressing AML cells.

### Example 7. In Vitro Studies Showing mIFNg and PEG mIFNg Increase Expression of MHC-I Proteins, MHC-II Proteins, and PD-L1 Proteins in Mouse Tumor Cells

A. Murine B16F10 melanoma cells were treated in vitro with 10 or 50 ng/ml of mIFNg (obtained from R&D Systems, Inc.) or the same concentration of PEG mIFNg for 72 hr. Samples of the cells were removed after 24, 48, or 72 h and analyzed by flow cytometry (FACS) for MHC-I, MHC-II and PD-L1 expression on the tumor cells. MHC-II expression was increased from <1% of cells to 80 to 100% of cells by both PEG-mIFNg and mIFNg. The percent of cells expressing MHC-I-H2Kb increased from about 20% to about 80 to 100% after mIFNg or PEG mIFNg treatment, and the percent of cells expressing PD-L1 increased from less than about 5% to about 75 to 90%. The relative level of MHC-II, MHC-I and PDL-1 expression, as measured by mean fluorescence intensity (MFI) also was dramatically increased after mIFNg and PEG mIFNg treatment. These changes were apparent after 24 h, generally peaked at 48 h, and decreased somewhat at 72 h.

B. Murine MLL-AF9 cells. (MLL-AF9 cells were created by transduction of C57BL/6 bone marrow cells with a MLL-AF9 MSCV construct—Krivtsov et al. (2006) Nature 442, 818-822). MLL-AF9 cells were treated in vitro with 10 or 50 ng/ml of mIFNg (obtained from R&D Systems, Inc.) or the same concentration of PEG mIFNg for 72 hr and analyzed by flow cytometry (FACS) for MHC-I, MHC-II and PD-L1 expression on the tumor cells. The percent of cells expressing MHC-II increased by 40% or more following treatment with PEG-mIFNg or mIFNg. In the absence of mIFNg proteins fewer than about 10% of the cells expressed MHC II. The expression of MHC-I-H2Kb and PD-L1 also increased following treatment with PEG mIFNg or mIFNg. The relative expression levels of these proteins, as measured by mean fluorescence intensity (MFI), were higher in cells treated with PEG mIFNg or mIFNg compared to no cytokine controls (vehicle). Most cells expressed MHC I with or without mIFNg treatment, but the relative intensity of MHC I expression was increased by greater than 3-fold in cells treated with mIFNg or PEG mIFNg. The percent of cells expressing PD-L1 was increased by at least 2-fold or more, and PD-L1 expression level (MFI) was higher in cells treated with mIFNg or PEG mIFNg. These changes were generally apparent by 24 or 48 h and persisted to 72 h.

C. Primary murine AML1 parent cell. (Dnmt3aR878H/FLT3-ITD AML1 tumors: the AML parental tumor line was made by transduction of C57BL/6 bone marrow cells from a Dnmt3aR878H knock-in mouse (the mouse version of human DNMT3AR882H mutation) with a FLT3-ITD MSCV construct; Smith et al., 2021, Nature Communications (2021) 12:4549; doi.org/10.1038/s41467-021-24800-7). AML1 cells were treated in vitro with 10 or 50 ng/ml of mIFNg (obtained from R&D Systems, Inc.) or the same concentration of PEG mIFNg for 72 hr and analyzed by flow cytometry for MHC-I, MHC-II and PD-L1 expression on the tumor cells. mIFNg and PEG mIFNg did not upregulate MHC-II expression in this parent murine AML tumor, as measured by the percent of cells expressing MHC-II or by relative expression level (MFI) of MHC-II per cell. Almost all cells expressed MHC I, but the relative expression level (MFI) of MHC-I-H2Kb increased by 2 to 3-fold in the cells after mIFNg or PEG mIFNg treatment. The percent of cells expressing PDL-1 was increased by 25 to 60% at 24 h and 48 h after treatment with mIFNg or PEG mIFNg, but PD-L1 MFI did not change appreciably after mIFNg or PEG mIFNg treatment.

D. AML1-Rel1—AlloTR. Allogenic T cell resistant tumor line. (The allogeneic T cell resistant (alloTR) AML1 tumors were made by serial hematopoietic stem cell transplantation with allogeneic T cells as described in Example 12). AML1-Rel1—AlloTR cells were treated in vitro with 10 or 50 ng/ml of mIFNg (obtained from R&D Systems, Inc.) or the same concentration of PEG mIFNg for 72 hr and analyzed by flow cytometry for MHC-I, MHC-II and PD-L1 expression on the tumor cells. mIFNg and PEG mIFNg did not upregulate MHC-II expression in this Allo T resistant murine AML tumor, as measured by percent MHC-II positive cells or by increase in MHC-II relative expression (MFI). Almost all cells expressed MHC-I H2Kb in the presence or absence of mIFNg or PEG mIFNg, but MHC-I-H2Kb expression level (MFI) increased by greater than 4-fold following treatment with mIFNg or PEG IFNg. The percent of cells expressing PD-L1 increased from about 90% to about 100% following treatment with mIFNg or PEG mIFNg, and the relative PD-L1 expression level per cell (MFI) increased 2- to 3-fold following treatment with mIFNg or PEG mIFNg. The increase in relative PD-L1 expression was greater than in the primary AML1 parent cell line.

### Example 8. PEG mIFNg Administration Increases Expression of MHC-I, MHC-II, and PD-L1 on Different Types of Blood Cells, and Causes Transient Changes in Blood Cell Numbers and Blood Cell Types in Mice

A. Changes in blood cell numbers in mice. C57BL/6 mice were given three times per week sc injections for two weeks (dosing every other day on Monday, Wednesday, Friday) of PEG mIFNg using doses of 15, 7.5 or 3.8 mcg/mouse/injection (about 0.75, 0.375 and 0.19 mg/kg/injection, which correspond to human equivalent doses of about 0.060, 0.030, and 0.015 mg/kg/injection, adjusted for body mass differences between mice and humans). All doses were well tolerated, with no significant effects on body weights or survival. Mice were bled weekly [on days 0 (pre-dose), and on days 7, 14, 21, and 28 post-dose) for flow cytometry (FACS) analysis of blood cell subtypes and expression of MHC-I, MHC-II, PD-L1, and CD119 (also known as IFNg receptor 1) proteins. Mice were sacrificed after 28 days and FACS analysis was performed on their bone marrow and spleen cells. Control mice were injected with the same volume of phosphate buffered saline (PBS).

The total white blood cell (WBC) count was similar in all mice prior to treatment, but WBC count decreased by 20% to 40% in the PEG mIFNg mouse groups compared to the PBS mouse group during the 2 week treatment phase (measured on days 7 and 14). WBC count was similar in the PEG IFNg groups and the PBS group on days 21 and 28.

NK cell numbers were similar in the treatment groups at day 0, but were about 3.5-fold higher in the PEG-mIFNg-treated mice on day 7 compared to PBS-treated mice. The NK cell increase stimulated by PEG-mIFNG was largely due to an approximate 6-fold increase in the number of Ly6C^{lo} NK cells on day 7. NK cell numbers were similar in all treatment groups on days 14, 21, and 28.

B cell numbers were similar between treatment groups at day 0, but about 50% lower in the PEG-mIFNg-treated mice on days 7 and 14 compared to PBS-treated mice. B cell numbers were similar in all treatment groups on day 21.

B. Changes in MHC-II expression on blood cells in mice. The percent MHC-II-positive monocytes increased about 3- to 4-fold in PEG mIFNg-treated mice compared to mice treated with PBS. The PEG-IFNg-stimulated increase was largely confined to Ly6C^{hi} monocytes, although Lys6C^{lo} myeloid cells also showed about a 1.5- to 2-fold increase in percent MHC-II positive cells.

PEG-mIFNg increased MHC-II expression on B cells by about 40 to 65% (as measured by mean fluorescence intensity) in the mice, which was apparent on days 7, 14, and 21 when compared to mice treated with PBS.

C. Changes in MHC-I expression on blood cells in mice. PEG-mIFNg treatment increased MHC-I expression on B cells by about 30 to 70%, Ly6C^{lo} myeloid cells by about 25 to 70%, monocytes by about 25 to 70%, and neutrophils by 300 to 500%, in mice compared to PBS, as measured by MFI increases.

D. Changes in PD-L1 expression on blood cells in mice. PEG-mIFNg increased PD-L1 expression on B cells by about 40 to 90%, T cells by about 25 to 90%, Ly6C^{lo} myeloid cells by about 25 to 90%, and neutrophils by about 50 to 400% in the mice compared to PBS, as measured by MFI increases. PEG mIFNg decreased PD-L1 MFI on day 14 by about 50% relative to PBS controls.

E. Changes in CD119 expression on blood cells in mice. Very few changes were noted for CD119 expression on B cells, T cells, Ly6C^{lo} monocytes, Ly6C^{hi} monocytes and neutrophils in blood of the mice after PEG-mIFNg treatment. PEG-mIFNg decreased CD119 expression on NK cells by about 50% on day 14 and 21.

F. Changes in bone marrow and spleen cell composition in mice. There were no significant differences in the composition of bone marrow cells (B cells, T cells, NK cells, Ly6C^{lo} monocytes, Ly6C^{hi} monocytes and neutrophils) in the PBS or PEG mIFNg mice 2 weeks after PEG-mIFNg treatment was stopped.

There were no significant differences in the levels of MHC-I expression, PD-L1 expression, or CD119 (IFNg receptor 1) expression among B cells, T cells, NK cells, Ly6C^{lo} myeloid cells, Ly6C^{hi} monocytes and neutrophils present in bone marrow of PBS-treated or PEG mIFNg-treated mice 2 weeks after PEG-mIFNg treatment was stopped.

There were no significant changes in the composition of spleen cells (B cells, T cells, NK cells, Ly6C^{lo} myeloid cells, Ly6C^{hi} monocytes and neutrophils in bone marrow of the PBS-treated or PEG mIFNg-treated mice two weeks after PEG-mIFNg treatment was stopped.

Relative MHC class I expression (MFI) was similar in B cells, T cells, and Ly6C^{hi} monocytes isolated from the spleens of PBS-treated or PEG mIFNg-treated mice 2 weeks after PEG-mIFNg treatment was stopped. MHC class I expression showed a small approximate 20 to 35% decrease in NK cells isolated from the spleens of PBS-treated or PEG mIFNg-treated mice 2 weeks after PEG-mIFNg treatment was stopped. There were no significant differences in the levels of PD-L1 expression, and CD119 expression, among B cells, T cells, Lys6C^{lo} myeloid cells, Lys6C^{hi} monocytes and neutrophils present in the spleens of PBS-treated or PEG mIFNg-treated mice two weeks after PEG-mIFNg treatment was stopped. There were a slight decrease in the relative level of PD-L1 expression and slight increase in the relative level of CD119 expression among NK cells present in the spleens of PBS-treated or PEG mIFNg-treated mice two weeks after PEG-mIFNg treatment was stopped.

### Example 9. Identification of an Optimum Time for PEG mIFNg Administration Following Lethal Irradiation and Hematopoietic Cell Transplant (HCT) in Mice

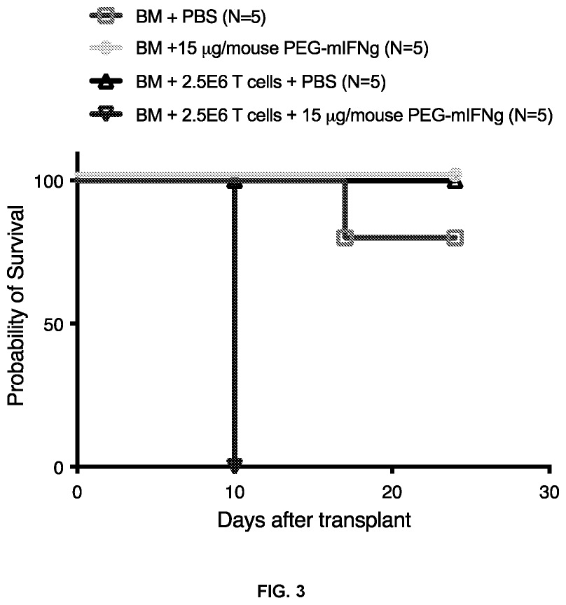

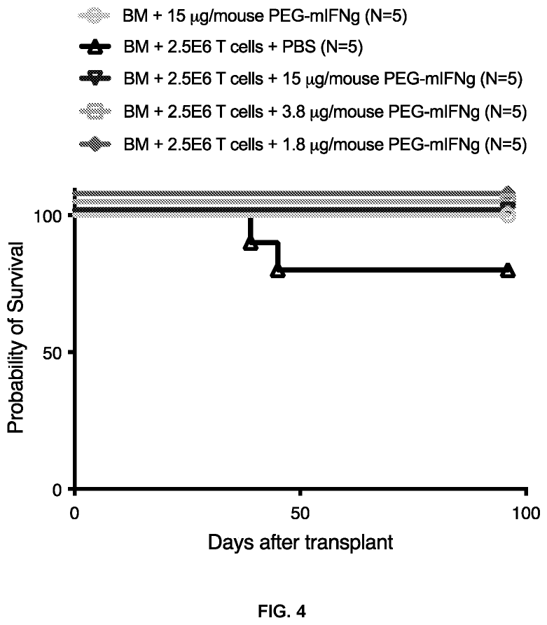

C57BL/6 (B6) mice were lethally irradiated (1200 cGy total body irradiation administered via two 600 cGy doses spread 4 hours apart) and given an intravenous injection of 4×10⁶ T cell depleted BALB/c bone marrow cells with or without 2.5×10⁶ allogeneic BALB/c donor T cells. On day 4 after transplant, mice received 3×/week subcutaneous injections of vehicle (PBS) or 15 mcg/mouse PEG-mIFNg. All mice transplanted without T cells and treated with PEG-mIFNg survived to 24 days. Likewise, all mice transplanted with allogeneic T cells and treated with vehicle survived to 24 days. In contrast, all mice transplanted with allogeneic T cells and treated with PEG-mIFNg died on day 10 after transplant. (FIG. 3) The inventors hypothesized that irradiation and hematopoietic cell transplantation created an inflammatory environment that was exacerbated by PEG mIFNg. Therefore, the experiment was repeated with the same design except that allogeneic T cell administration and PEG mIFNg (and PBS) treatment was delayed until day 10 following irradiation and HCT. Mice received subcutaneous injections of PBS or 15, 3.8, or 1.8 μg per mouse of PEG mIFNg on days 10, 12, 14, 16, 18, and 20 after transplant. All mice treated with allogeneic T cells and PEG mIFNg beginning on day 10 survived to study end (day 96) following this delayed treatment protocol (FIG. 4). This result indicates that PEG IFNg treatment should be delayed to at least day 5 and preferably until day 10 or later after irradiation and HCT.

### Example 10. General Method for Screening Tumor Cell Lines and Patient Tumor Cells In Vitro for Increased MHC-I, MHC-II and PD-L1 Expression in Response to IFNg Treatment

Tumor cell lines and tumor cells isolated from an animal can be screened to determine if they increase expression of MHC-I, MHC-II, and PD-L1 proteins (as well as other checkpoint inhibitor molecules and T cell agonist and antagonist molecules) in response to IFNg and PEG IFNg treatment by growing the cells in vitro for 24 to 72 h in media containing 10 to 50 ng/mL IFNg or PEG IFNg, as described in Example 8. As a control, the tumor cells can be grown in the same media without IFNg or PEG IFNg. At different time points the cells can be stained with fluorescently-labeled antibodies specific for MHC-I, MHC-II, or PD-L1, and analyzed by FACS as described in Example 8. Increased IFNg-stimulated or PEG IFNg-stimulated MHC-I, MHC-II, or PD-L1 expression can be demonstrated by an increase in the percent of cells expressing MHC-I, MHC-II, or PD-L1 relative to untreated cells or cells treated with media only, or by an increase in the MFI of IFNg-treated cells or PEG IFNg-treated cells expressing MHC-I, MHC-II, or PD-L1 compared to untreated cells or cells treated with media only. Alternatively, RNA can be isolated from tumor cells treated in vitro with and with IFNg or PEG IFNg and analyzed using qPCR or RNAseq methods to determine whether they increase RNA levels for MHC-I, MHC-II, and PD-L1 proteins, as well as for other proteins involved with antigen processing, antigen presentation, checkpoint inhibitor proteins and T cell agonist and antagonist proteins).

### Example 11. Use of PEG IFNg and IFNg Alone and in Combination with Other Cancer Therapeutics to Inhibit Tumor Growth and/or to Stimulate an Anti-Tumor Immune Response in an Animal

PEG IFNg and IFNg may be used to inhibit growth of a tumor in an animal and/or to stimulate an anti-tumor immune response in an animal by administering one or more effective doses of the proteins alone or in combination with one or more effective doses of a different anti-cancer therapeutic. Examples of anti-cancer therapeutics include but are not limited to checkpoint inhibitor antibodies such as anti-PD-1, anti-PD-L1, anti-CTLA4 and anti-TIGIT antibodies, T cell agonist proteins, anti-cancer drugs, chemotherapeutic agents, radiation, engineered T cells, including chimeric antigen receptor (CAR)-T cells, engineered monocytes or macrophages including CAR-monocytes and CAR-macrophages, engineered NK cells, including CAR NK cells, and HCT.

An animal with a tumor can be administered one or more effective doses of an IFNg analog such as PEG IFNg in order to increase expression of MHC-I, MHC-II and/or PD-L1 in the tumor cells or in the animal's cells. Optionally, the animal is also administered a different anti-cancer therapeutic such as those described above. The general methods described below can be used to show how mIFNg and PEG mIFNg can be used alone to inhibit tumor growth in immune competent mice. The methods also can be used to show how PEG IFNg (or IFNg) can be used in combination with a different anti-cancer therapeutic (e.g., an anti-PD-L1 mAb) to additively or synergistically inhibit growth of murine B16F10 melanoma cells, murine A20 lymphoma cells, and murine CT26 colon cancer cells in immune competent mice. Other murine tumor cells can be tested in the same manner. These and other murine and human tumor cell lines are available from the American Type Culture Center (Manassa, VA). PEG IFNg is used as a representative IFNg analog, but other IFNg analogs can be tested in the same manner.

Murine B16F10 melanoma cells may be transplanted into immune competent, syngeneic C57BL/6 mice. After tumor transplantation, mice can be treated with one or more effective doses or PEG mIFNg alone or in combination with one or more effective doses of an anti-PD-L1 antibody, preferably an anti-PD-L1 mAb such as 10F.962 mAb (available from Bio X Cell, Lebanon, NH). Control mice should receive injections of equivalent volumes of a vehicle (control) solution such as phosphate buffered saline or the protein's formulation buffered. An isotype matched antibody from the same species may be used as a control for the anti-PD-L1 antibody. Tumor growth (tumor volume) can be measured over the next 20 to 70 days using a formula of volume=(Length×Width×Width/2). PEG IFNG or IFNg alone may or may not inhibit growth of the tumors relative to vehicle solution. Anti-PD-L1 antibody alone may or may not inhibit growth of the tumor compared to vehicle. The combination of PEG IFNg (or IFNg) and the anti-PD-L1 antibody will demonstrate additive or synergistic inhibition of tumor growth if tumor growth inhibition is greater with the combination of the two drugs than with PEG IFNg alone (or IFNg alone) or anti-PD-L1 alone. Mice that survive and eradicate the tumor may be retransplanted at a later time point with additional B16F10 cells to test whether the mice develop a memory immune response to the tumor. Mice that reject the retransplanted tumor cells will have demonstrated a memory immune response to the tumor.

Murine A20 lymphoma cells and murine CT26 colon tumor cells may be tested in the same manner as the B16F10 tumor cells except that they should be transplanted into immune competent syngeneic Balb/c mice.

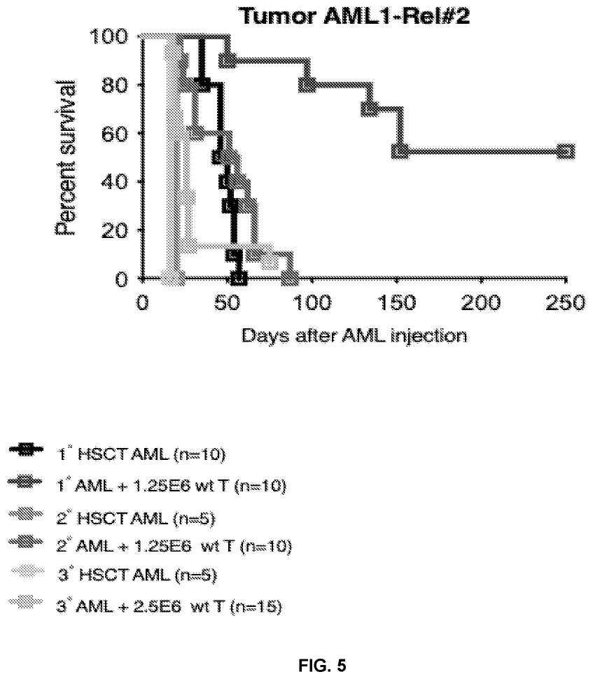

Example 12.: The experiments in this Example demonstrate that PEG mIFNg is able to re-sensitize allogeneic T cell resistant murine AML tumor cells to allogeneic T cell killing in mice. The studies used murine AML cells (Dnmt3a^{R878H}/FLT3-ITD model; described in Example 7), which were genetically modified to express green fluorescent protein (GFP). Allogeneic T cell resistant murine AML cells (alloT^{r} AML1, also referred to as AML1-alloT^{r}) were generated via sequential MHC-mismatched allo-hematopoietic stem cell transplants (HSCTs). Lethally irradiated C57BL/6 (B6) recipient mice were transplanted with syngeneic B6 AML1 tumors and allogeneic T cell depleted bone marrow (BM) from 129 SVJ mice in the presence or absence (BM only) of allogeneic wild type T cells from 129 SVJ mice. AML cells were harvested from relapsing mice and re-transplanted into new B6 recipient mice in serial transplantation. In vivo allogeneic T cell resistance was demonstrated by the AML cells treated with allo T cells (also referred to as donor lymphocytes, donor T lymphocytes, or donor T cells) in a tertiary transplant in mice dying at about the same time as the no T cell treated control mice. As shown in FIG. 5 and Table 3, mice injected with the AML1 tumor cells and allogeneic T cells displayed a significantly decreased median survival time (MST) from >250 days (undefined) to 29.9 days during progression from the primary (1°) allo-HSCT to the tertiary (3°) allo-HSCT, respectively (p<0.0001, 1° HSCT vs. 3° HSCT). Genomic analysis using whole exome sequencing showed no mutations in MHC I, MHC II or genes associated with immune functions in the alloT^{r} AML1 cells. Preliminary transcriptome analysis of the alloT^{r} AML1 after the tertiary transplant showed down regulation of genes mediating cell-cycle regulation and activation of T cells, including regulation of IFNg production pathways.

TABLE 3
Generation of a T cell resistant AML cell line by serial transplantation
in mice in the presence of wild type T cells

| | | GVHD | AML | MST |
|---|---|---|---|---|
| Treatment | # Mice | deaths | deaths | (days) |
| 1° HSCT AML | 10 | 0 | 5 | 46 |
| 1° AML + 1.25 × 10⁶ | 10 | 2 | 2 | >250 |
| wild type T cells | | | | |
| 2° HSCT AML | 5 | 0 | 5 | 20 |
| 2° AML + 1.25 × 10⁶ | 10 | 2 | 7 | 52.8 |
| wild type T cells | | | | |
| 3° HSCT AML | 5 | 0 | 5 | 16 |
| 3° AML + 2.5 × 10⁶ | 15 | 0 | 13 | 29.9 |
| wild type T cells | | | | |

GVHD (Graft Versus Host Disease)
AML (Acute Myelogenous Leukemia)
MST (Median Survival Time)
HSCT (hematopoietic stem cell transplant)

To evaluate whether PEG-mIFNg could re-sensitize the alloT^{r} AML1 cells to allogeneic T cell killing after allo-HSCT, the inventors reconstituted lethally irradiated B6 recipient mice with 129 SVJ bone marrow (T cell depleted) alone on day 0. On day 10 after allo-HSCT, mice were injected intravenously with 1.5×10⁶ alloT^{r} AML cells into the tail vein and divided into the following groups: (1) untreated controls (T cell depleted bone marrow only), (2) PEG-mIFNg only, (3) 5×10⁶ allogeneic 129 SVJ T cells (allo-T; also referred to as donor lymphocytes, donor T lymphocytes, or donor T cells) only on day 10, or (4) 5×10⁶ allogeneic 129 SVJ T cells on day 10 plus PEG-mIFNg. Two doses of PEG-mIFNg (15 ug or 3.8 ug per injection) were tested in these experiments and treatment was started on day 10 post-allo-HSCT at the same time as allo-T injection. A total of 6 PEG-muIFNg doses were given (3 doses/week×2 weeks). PEG mIFNg was given by subcutaneous (sc) injection. Mice were monitored for survival and graft-versus-host disease (GVHD). alloT^{r} AML1 tumor burden was assessed by fluorescence activated cell sorting (FACS) and using green fluorescent protein (GFP) expression as a measure tumor cell burden.

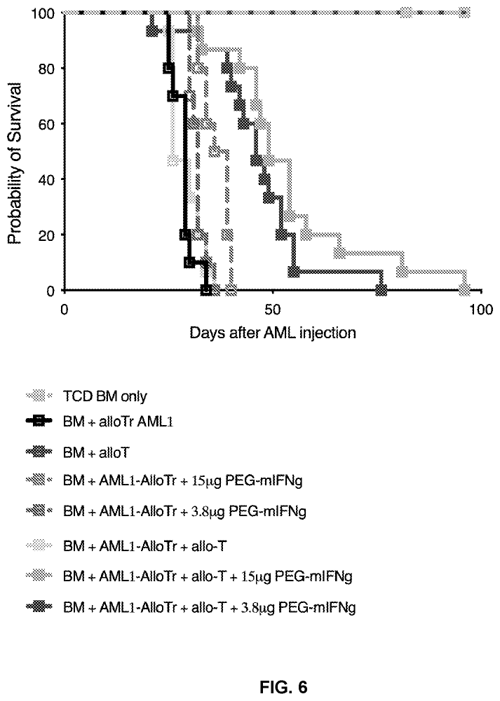

The results of this experiment were that the untreated T cell depleted bone marrow only control mice all died of AML within 18 days (MST of 29 days) of tumor injection (FIG. 6 and Table 4). Treatment with PEG-mIFNg alone slightly extended the MST of mice in a dose dependent manner with 15 ug PEG-mIFNg dosing providing a MST of 38 days while the 3.8 ug dose level exhibited a MST of 32 days. Recipient mice treated with 5×10⁶ allo-T cells alone succumbed to leukemia (AML) similar to the untreated controls (MST of 26 days), confirming that the alloT^{r} AML cells were resistant to allo-T cell killing. However, mice treated with allo-T cells+15 ug PEG-mIFNg exhibited significantly prolonged (extended) survival (MST of 54 days) compared to the allo-T cells only (MST of 26 days; p=0.0001) and the 15 ug PEG-mIFNg only controls (MST of 38 days; p=0.0001). Mice treated with allo-T cells+3.8 ug PEG-mIFNg exhibited significantly prolonged (extended) survival (MST of 46 days) compared to the allo-T cells only (MST of 26 days; p=0.0001) and the 3.8 ug PEG-mIFNg only controls (MST of 32 days; p=0.0001). The 15 ug PEG mIFNg dose level provided greater graft-versus-leukemia (GVL) activity (only 9/15 mice relapsed (i.e., died from AML) vs. 13/15 with 3.8 ug PEG IFNg dosing), but was associated with slightly higher GVHD (6/15 mice developed lethal GVHD). In contrast, the 3.8 ug PEG-mIFNg dose had fewer GVHD related deaths (2/15) but provided slightly less GVL activity (13/15 mice succumbed to AML).

TABLE 4
Improved survival of mice with allo-T resitant
AML1-alloT^{r} tumors treated with a combination of PEG mIFNg
and allo T cells compared to PEG mIFNg alone or allo-T cells alone.

| | # | GVHD | AML | MST |
|---|---|---|---|---|
| Treatment | Mice | deaths | deaths | (days) |
| BM only | 10 | 0 | — | N/A |
| BM + AML1-alloT^{r} | 10 | — | 10/10 | 29 |
| BM + allo-T | 10 | 0/10 | — | N/A |
| BM + AML1-AlloT^{r} + | 10 | 0/10 | 10/10 | 38 |
| 15 μg PEG mIFNg | | | | |
| BM + AML1-AlloT^{r} + | 10 | 0/10 | 10/10 | 32 |
| 3.8 μg PEG mIFNg | | | | |
| BM + AML1-AlloT^{r} + | 15 | 0/15 | 15/15 | 26 |
| allo-T | | | | |
| BM + AML1-AlloT^{r} + | 15 | 6/15 | 9/15 | 54 |
| allo-T + 15 μg PEG mIFNg | | | | |
| BM + AML1-AlloT^{r} + | 15 | 2/15 | 13/15 | 46 |
| allo-T + 3.8 μg PEG mIFNg | | | | |

BM, bone marrow, T cell depleted
Allo-T, allogeneic T cells
AML1-AlloT^{r}, allogeneic T cell resistant AML1 tumor cells
GVHD (Graft Versus Host Disease
AML (Acute Myeloid Leukemia)
MST (Median Survival Time)
N/A, Not applicable, no deaths

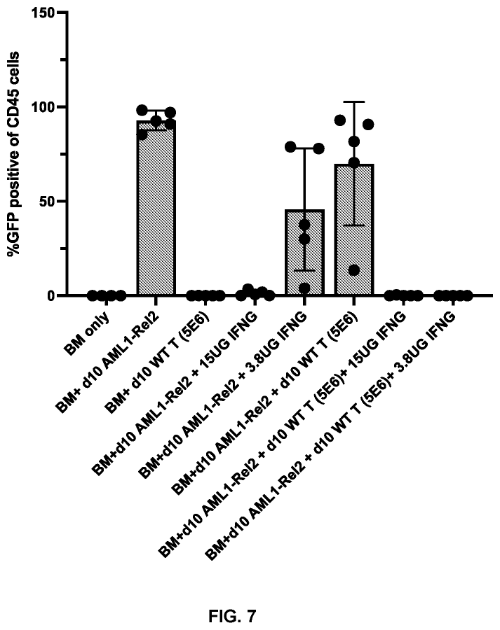

On day 30 flow cytometry was done on peripheral blood CD45 positive cells of the different treatment groups, measuring % GFP positive cells as an indicator of AML1 tumor blast burden. The results showed that GFP positive AML blasts were significantly reduced or undetectable in peripheral blood of mice treated with PEG mIFNg plus allo-T cells compared to mice treated with allo-T cells only, PEG IFNg only, or vehicle only (FIG. 7).

This study demonstrates that treatment with PEG-mIFNg beginning 10 days after allo-HSCT can re-sensitize the alloT^{r} AML1 tumors to killing by allogeneic T cells (allo-T cells; also referred to as donor lymphocytes, donor T lymphocytes, or donor T cells) and provides a significant survival advantage compared to allo-T cell treatment alone or PEG mIFNg treatment alone. Extending PEG-mIFNg treatment using the 3.8 ug or lower dose beyond 2 weeks may enhance GVL activity while limiting lethal GVHD. PEG mIFNg doses lower than 3.8 ug, such as from 0.01 to 3.8 ug, can be tested using similar methods to determine whether they also re-sensitize the alloT^{r} AML1 tumors to allogeneic T cell-mediated killing and extend survival. The allo-T cells were given at the same time as PEG-mIFNg treatment began in this experiment, but also could be given to the animal after PEG mIFNg treatment or before PEG mIFNg treatment.

While various embodiments of the present invention have been described in detail, it is apparent that modifications and adaptations of those embodiments will occur to those skilled in the art. It is to be expressly understood, however, that such modifications and adaptations are within the scope of the present invention.

### REFERENCES

Christopher M J, Petti A A, Rettig M P et al (2018) Immune escape of relapsed AML cells after allogeneic transplantation. N Engl J Med. 379 (24): 2330-2341. doi: 10.1056/NEJMoa1808777.
Fam C M, Eisenberg S P, Carlson S J, et al. (2014) PEGylation improves the pharmacokinetic properties and ability of interferon gamma to inhibit growth of a human tumor xenograft in athymic mice. Journal of Interferon and Cytokine Research 34:759-768.
Forero A, Li Y, Chen D et al. (2016) Expression of the MHC class II pathway in triple negative breast cancer tumor cells is associated with a good prognosis and infiltrating lymphocytes. Cancer Immunol Res 4:390-399.
FDA CDER (2005) Guidance for industry: Estimating the maximum safe starting dose in initial clinical trials for therapeutics in adult healthy volunteers (available on FDA CDER website).
Jackson, S H, Miller G F, Segal B H, Mardiney M 3rd, Domachowske, J B, Gallin, J I, and Holland S M. (2001) J Interferon Cytokine Res. 21 (8): 567-73. IFN-gamma is effective in reducing infections in the mouse model of chronic granulomatous disease (CGD).
Samuel C E, Knutson G S (1982) Mechanism of interferon action. Kinetics of decay of the antiviral state and protein phosphorylation in mouse fibroblasts with natural and cloned interferons. J Biol Chem 257:11596-1180.
Wall L, Burke F, Smyth J, Balkwill F (2003) The anti-proliferative activity of interferon gamma on ovarian cancer: In vitro and in vivo. Gynecol Oncol 88:149-151.
Schroder K, Hertzog P J, Ravasi T, Hume D A (2004) Interferon gamma: an overview of signals, mechanisms and functions. J Leukoc Biol 75:163-189.
Pace, J., Russell, S. LeBlanc, P., Murasko, D. (1985) Comparative effects of various classes of mouse interferons on macrophage activation for tumor cell killing. J. Immunol. 134:977-981.
Liang Y, Tang H, Guo J et al (2018) Targeting IFN alpha to tumor by anti-PD-L1 creates feedforward antitumor responses to overcome checkpoint blockage resistance. Nature Communications (2018) 9:4586 DOI: 10.1038/s41467-018-06890-y.
Chen S, Crabill G A, Pritchard D S et al. (2019) Mechanisms regulating PD-L1 expression on tumor and immune cells. Journal for ImmunoTherapy of Cancer (2019) 7:305. (doi.org/10.1186/s40425-019-0770-2).
Mills B N, Connolly K A, Ye J, et al. (2019) Stereotactic body radiation and interleukin-12 combination therapy eradicates pancreatic tumors by repolarizing the immune microenvironment. Cell Reports 29:406-421.
Cai X, Wei B, Li L et al. (2020) Apatinib enhanced anti-PD-1 therapy for colon cancer in mice via promoting PD-L1 expression. International Immunopharmacology 88 (2020) 106858. (doi.org/10.1016/j.intimp.2020.106858).
Frasca L, Nasso M, Spensieri F et al (2008) IFN gamma arms human dendritic cells to perform multiple effector functions. J Immunology 180:1471-1481.
Curran M A, Montalvo W, Yagita H, Allison J P (2010) PD-1 and CTLA-4 combination blockade expands infiltrating T cells and reduces regulatory T and myeloid cells within B16 melanoma tumors PNAS USA 107:4275-4280.
Bourgeois-Daigneault M-C, Roy D G, Falls T et al. (2016) Oncolytic vesicular stomatitis virus expressing interferon-gamma has enhanced therapeutic activity. Oncolytics 3, 16001; doi: 10.1038/mto.2016.1.
Krivtsov, A., Twomey, D., Feng, Z. et al. (2006) Transformation from committed progenitor to leukaemia stem cell initiated by MLL-AF9. Nature 442, 818-822. (doi.org/10.1038/nature04980).
Toffalori, C., Zito, L., Gambacorta, V. et al. (2019) Immune signature drives leukemia escape and relapse after hematopoietic cell transplantation. Nature Medicine 25, 603-611. (doi.org/10.1038/s41591-019-0400-z).
Smith, A. M., La Valle, T. A., Shinawi M. et al. (2021), Functional and epigenetic phenotypes of humans and mice with DNMT3A Overgrowth Syndrome. Nature Communications (2021) 12:4549.

What is claimed is:

1. A method for inhibiting growth of a tumor in an animal having a tumor, the method comprising administering to the animal one or more effective doses of an interferon gamma (IFNg) analog, wherein the IFNg analog increases tumor cell major histocompatibility complex (MHC-II) protein expression, tumor cell major histocompatibility complex (MHC-I) protein expression and/or tumor cell programmed death ligand 1 (PD-L1) protein expression.

2. The method of claim 1, wherein the animal is further administered one or more effective doses of an anti-PD-L1 antibody, an anti-programmed cell death protein 1 (PD-1) antibody, a checkpoint inhibitor antibody, or a T cell agonist drug.

3. The method of claim 2, wherein the checkpoint inhibitor antibody is selected from the group consisting of an anti-cytotoxic T-lymphocyte Antigen 4 antibody (anti-CTLA4 antibody), an anti-lymphocyte-activator gene 3 antibody (anti-LAG-3 antibody), an anti-T-cell immunoglobulin mucin-3 antibody (anti-TIM 3 antibody), an anti-tissue inhibitor of matrix metalloproteinase antibody (anti-TIMP antibody), and an anti-T-cell immunoreceptor with Ig and ITIM domains antibody (anti-TIGIT antibody).

4. The method of claim 2, wherein the T cell agonist protein is a protein drug selected from the group consisting of an OX-40 agonist, a 4-1BB agonist, and a CD40 agonist.

5. The method of claim 1, wherein the animal is further administered one or more effective doses of an anti-PD-L1 antibody or an anti-PD-1 antibody, wherein the IFNg analog induces expression of PD-L1 in the tumor cell, and wherein tumor growth inhibition with the combination of the IFNg analog and the anti-PD-L1 antibody or the anti-PD-1 antibody is greater than the tumor growth inhibition obtained by treatment with the IFNg analog alone or the anti-PD-L1 antibody alone or the anti-PD-1 antibody alone.

6. A method for stimulating an anti-tumor immune response in an animal having a tumor, the method comprising administering to the animal one or more effective doses of an IFNg analog.

7. The method of claim 6, wherein the animal is further administered one or more effective doses of an anti-PD-L1 antibody or an anti-PD-1 antibody, wherein the IFNg analog induces expression of PD-L1 in tumor cells of the animal, and wherein the anti-tumor immune response obtained with the combination of the IFNg analog and the anti-PD-L1 antibody or anti-PD-1 antibody is greater than the anti-tumor immune response obtained by treatment with the IFNg analog alone or the anti-PD-L1 antibody alone or the anti-PD-1 antibody alone.

8. The method of claim 6, wherein the IFNg analog increases expression of PD-L1, MHC Class II proteins, and/or MHC Class I proteins in tumor cells of the animal.

9. The method of claim 6, wherein the animal is further administered one or more effective doses of an anti-PD-L1 antibody, an anti-PD-1 antibody, a checkpoint inhibitor antibody, or a T cell agonist protein.

10. The method of claim 9, wherein the checkpoint inhibitor antibody is selected from the group consisting of an anti-CTLA4 antibody, an anti-LAG-3 antibody, an anti-TIM 3 antibody, an anti-TIMP antibody, and an anti-TIGIT antibody.

11. The method of claim 9, wherein the T cell agonist protein is a protein drug selected from the group consisting of an OX-40 agonist, a 4-1BB agonist, and a CD40 agonist.

12. A method for stimulating a graft versus leukemia anti-tumor immune response in an animal comprising administering to an animal with a leukemia an IFNg analog that stimulates increased expression of MHC-II proteins or MHC-I proteins in the leukemia cells of the animal.

13. A method for stimulating a graft versus leukemia anti-tumor immune response in an animal comprising modifying the animal's leukemia cells with a gene encoding class II transactivator (CIITA).

14. The method of claim 12 or 13, wherein the animal is further administered one or more effective doses of an anti-PD-L1 antibody, an anti-PD-1 antibody, a checkpoint inhibitor antibody, or a T cell agonist protein.

15. The method of claim 14, wherein the checkpoint inhibitor antibody is selected from the group consisting of an anti-LAG-3 antibody, an anti-TIM 3 antibody, an anti-TIMP antibody, and an anti-TIGIT antibody.

16. The method of claim 14, wherein the T cell agonist protein is a protein drug selected from the group consisting of an OX-40 agonist, a 4-1BB agonist, and a CD40 agonist.

17. The method of any one of claims 1-16, wherein the tumor is selected from the group consisting of leukemia, lymphomas, myelomas, glioblastomas, melanomas, lung cancers, kidney cancers, liver cancers, pancreatic cancers, breast cancers, triple negative breast cancers, colon cancers, bladder cancers, fibrosaracoma cancers, gastric cancers, esophageal cancers, prostate cancers, and bone cancers.

18. The method of claim 17, wherein the leukemia is acute myelogenous leukemias (AML).

19. The method of any one of claims 1-18, wherein the animal is further administered an additional therapy selected from the group consisting of chemotherapeutic agents, anti-cancer drugs, checkpoint inhibitor antibodies, checkpoint inhibitor monoclonal antibodies, cell therapies, donor immune cells, donor lymphocytes, donor T lymphocytes, donor white blood cells, donor macrophages, donor NK cells, donor B cell lymphocytes, donor dendritic cells, engineered anti-tumor T cell therapies, engineered anti-tumor macrophage cell therapies, anti-tumor NK cell therapies, anti-tumor B cell therapies, anti-tumor dendritic cell therapies and combinations thereof.

20. The method of claim 17, wherein the tumor is resistant to anti-PD-1 antibody therapy or anti-PD-L1 antibody therapy.

21. The method of claim 17, wherein the tumor is a T cell resistant tumor.

22. The method of claim 17, wherein the tumor is a relapsed tumor.

23. The method of claim 22, wherein the relapsed tumor is a relapsed AML tumor.

24. The method of claim 22, wherein the relapsed tumor expresses lower levels of one or more proteins selected from the group consisting of MHC-I proteins, MHC-II proteins, and PD-L1 proteins after relapse compared to prior to relapse.

25. A method of increasing the number of natural killer (NK) cells in an animal, comprising administering to the animal one or more effective doses of an IFNg analog.

26. The method of any one of claims 1-25, wherein the IFNg analog is a long-acting IFNg analog, wherein the long acting INFg analog is modified with a polyethylene glycol (PEG).

27. The method of claim 26, wherein the INFg analog is modified with a PEG at a lysine residue of the protein.

28. The method of claim 26, wherein the INFg analog is modified with a PEG at the amino-terminus of the protein.

29. The method of claim 26, wherein the INFg analog is modified with a PEG at a sugar group in the protein.

30. The method of any one of claims 1-25, wherein the IFNg analog is an isolated cysteine variant of human IFNg (SEQ ID NO:1), wherein a cysteine residue is substituted for at least one amino acid selected from the group consisting of: D63, Q64, S65, I66, Q67, V99, V100, T101, D102 and L103.

31. The method of claim 30, wherein the isolated cysteine variant further comprises deletion or substitution of Q1 of SEQ ID NO:1 by a non-glutamine amino acid, and/or deletion or substitution of D2 of SEQ ID NO:1 by a non-aspartic amino acid.

32. The method of claim 30 or 31, wherein the variant is modified with a cysteine reactive moiety, wherein said cysteine reactive moiety is a PEG.

33. The method of any one of claims 1-25, wherein the INFg analog is a cysteine variant of human IFNg (SEQ ID NO:1) comprising at least one cysteine residue inserted in at least one region of IFNg selected from the group consisting of: the region preceding the first amino acid of SEQ ID NO:1, and the region following the last amino acid of SEQ ID NO:1.

34. The method of any one of claims 1-25, wherein the IFNg analog is a cysteine variant of human IFN-g (SEQ ID NO:1) comprising at least one cysteine residue substituted for at least one amino acid located in the C-D loop of IFN-g.

35. The method of claim 34, wherein the IFNg analog further comprises at least one cysteine residue substituted for at least one amino acid located in the region preceding helix A of IFNg.

36. The method of claim 34, wherein the IFNg analog further comprises at least one cysteine residue substituted for at least one amino acid located in the B-C loop of IFNg.

37. The method of claim 34, wherein the IFNg analog further comprises at least one cysteine residue substituted for at least one amino acid located in the D-E loop of IFNg.

38. The method of any one of claims 34-37, wherein the variant is modified with a cysteine reactive moiety, wherein said cysteine reactive moiety is a PEG.

39. The method of any one of claims 1-25, wherein the IFNg analog is human IFNg (SEQ ID NO:1) or an analog of human IFNg (SEQ ID NO:1).


SEQUENCE LISTING (2 sequences)

SEQ ID NO: 1 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 2 - protein, 133 aa, Mus musculus
HGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC